(19) United States
(12) Patent Application Publication
(10) Pub. No.: US 2024/0041820 A1
(43) Pub. Date: Feb. 8, 2024

(54) SENOTHERAPEUTIC COMPOUND

(71) Applicant: BIONEXA S.R.L., Bari (BA) (IT)

(72) Inventors: Mario CORREALE, Bari (BA) (IT); Giovanni GAMBERA, Francofonte (SR) (IT); Giovanni Mario PITARI, Augusta (SR) (IT); Venera RUSSO, Mascali (CT) (IT)

(21) Appl. No.: 18/254,957

(22) PCT Filed: Nov. 30, 2021
(86) PCT No.: PCT/IB2021/061106

§ 371 Date: May 30, 2023

(30) Foreign Application Priority Data
Dec. 1, 2020 (IT) 102020000029225

(51) Int. Cl. A61K 31/352; A61K 47/54; A61P 39/00

(52) U.S. Cl. CPC A61K 31/352; A61K 47/54; A61P 39/00

(57) ABSTRACT

A senotherapeutic compound including a synthetic derivative of flavones, according to the formula (I) as indicated below:

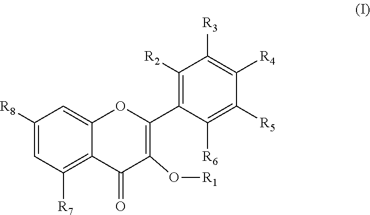

in which at least two of R2-R8 are H, and the remainder are individually selected from: H, OH, R1, OR1, NO2, NH2, NHR1, F, Cl, Br, I, where R1 is a radical.

The present invention relates to a senotherapeutic compound of the type specified in the preamble of the first claim.

A class of drugs known as senotherapeutics has recently been developed, including, in particular, senolytics and also senomorphic and senostatic drugs.

The senolytics are substances which, when taken by a user, selectively kill senescent cells in the human or animal body. Senomorphic and senostatic act on the secretions of senescent cells and block the senescence process.

Senescent cells are cells that are no longer able to divide and multiply. They are also subject to loss of physiological function, resistance to apoptosis and various cellular changes.

In addition, senescent cells contribute to the phenotype of aging, including frailty syndrome, sarcopenia and diseases associated with aging. Senescent astrocytes and microglia contribute to neurodegeneration.

The goal of senotherapeutics, in particular of senolytics, is therefore to delay, prevent, alleviate or reverse age-related diseases by eliminating, as selectively as possible, senescent cells.

Senolytic compounds have been studied for example by the Mayo Foundation for Medical Education and Research (Minnesota-US) for example in patent applications WO2015116735A1, WO2019183282A1 and US2015296755A1. Other senolytic compounds have been developed by the company Unity Biotechnology (California, US), for example in patent applications WO2019241567A1, US2019330199A1 and CA3043103A1.

However, the demand for more precise, performing or cheaper senotherapeutics is increasing.

In this situation, the technical task underlying the present invention is to devise a senotherapeutic compound, capable of substantially obviating at least part of the aforementioned drawbacks.

Within the scope of said technical task, it is an important object of the invention to obtain a senotherapeutic compound which functions selectively on senescent cells or on their secretions.

Another important technical task is to make a senotherapeutic compound whose production is economical.

The technical task and the specified aims are achieved by a senotherapeutic compound as claimed in the attached claim 1.

Examples of preferred embodiment are described in the dependent claims.

The characteristics and advantages of the invention are clarified below by the detailed description of preferred embodiments of the invention, with reference to the accompanying drawings, in which:

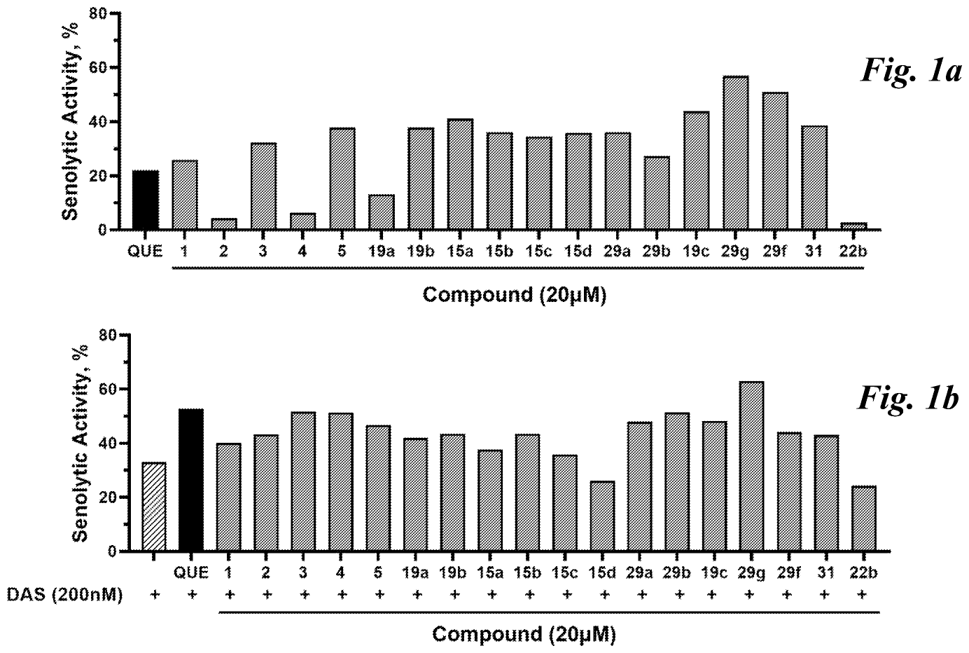

FIG. 1a shows a first graph showing the results obtained with the compound according to the invention;

FIG. 1b shows a second graph showing the results obtained with the compound according to the invention,

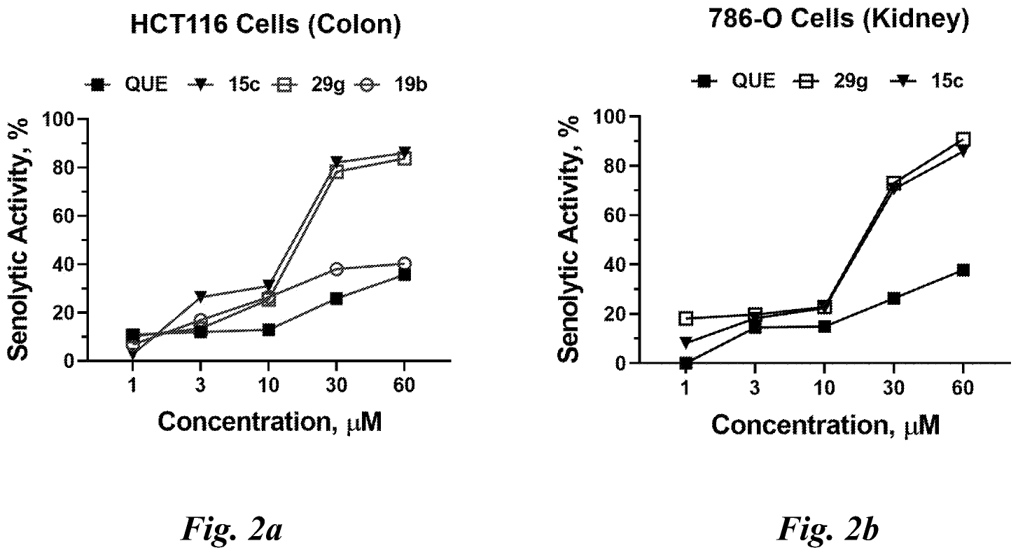

FIG. 2a shows a third graph showing the results obtained with the compound according to the invention;

FIG. 2b shows a fourth graph showing the results obtained with the compound according to the invention.

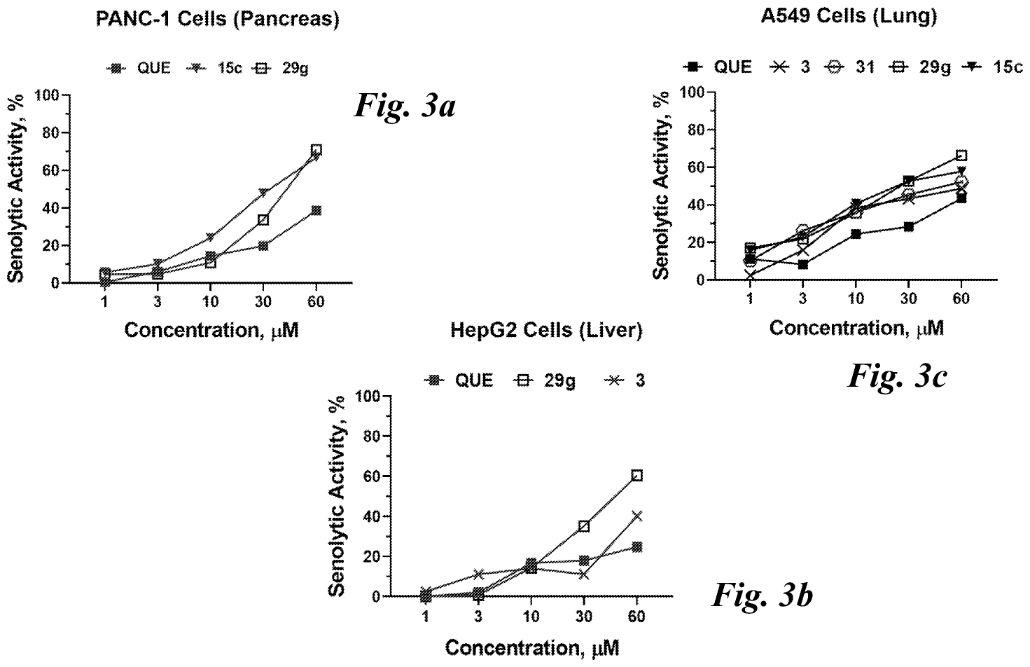

FIG. 3a shows a fifth graph showing results obtained with the compound according to the invention;

FIG. 3b shows a sixth graph showing results obtained with the compound according to the invention; and

FIG. 3c shows a seventh graph showing results obtained with the compound according to the invention.

In the present document, the measurements, values, shapes and geometric references (such as perpendicularity and parallelism), when associated with words like “about” or other similar terms such as “approximately” or “substantially”, are to be considered as except for measurement errors or inaccuracies due to production and/or manufacturing errors, and, above all, except for a slight divergence from the value, measurements, shape, or geometric reference with which it is associated. For instance, these terms, if associated with a value, preferably indicate a divergence of not more than 10% of the value.

Moreover, when used, terms such as “first”, “second”, “higher”, “lower”, “main” and “secondary” do not necessarily identify an order, a priority of relationship or a relative position, but can simply be used to clearly distinguish between their different components.

The measurements and data reported in this text are to be considered, unless otherwise indicated, as carried out in the ICAO International Standard Atmosphere (ISO 2533).

The senotherapeutic compound according to the invention is a substance for medical purposes for the treatment, preferably as selective as possible, of senescent cells.

The senotherapeutic compound preferably has a senolytic action and is therefore a senolytic compound. The senolytic compound is used to eliminate, preferably selectively, senescent cells.

The senotherapeutic compound can alternatively have a senostatic action, i.e. an action that blocks the senescence process.

The senotherapeutic compound can have, alternatively still, a senomorphic action, that is an action on the secretions of senescent cells.

Sinotherapeutics are therapeutic agents and methods that specifically target senescent cells, including their molecules and intracellular processes, and their released secretory substances. Senescent cells exhibit a unique and altered cell phenotype that arises in all tissues of an organism (including humans) as a consequence of many biological stressors. Among others, cellular senescence can be associated with aging and age-related diseases.

Sinotherapeutics can be further classified into at least two main categories:
Senolytics: agents that specifically eliminate senescent cells. Senolytics can eliminate senescent cells by inducing specific cell death mechanisms, including apoptosis, autophagy, necrosis, necroptosis or other forms of non-apoptotic programmed cell death (such as ferroptosis, pyroptosis, etc.). In some configurations, senolytics can target survival and anti-apoptotic pathways in senescent cells, known as senescent cell anti-apoptotic (SCAP) pathways.
Senomorphic: agents that specifically suppress the phenotype of senescent cells, without necessarily eliminating or killing senescent cells. Senomorphics modulate the functions and morphology of senescent cells, thus potentially delaying/preventing/inhibiting their formation, accumulation and pathological actions. In some configurations, the senomorphic includes inhibitors of the secretory associated senescence phenotype (SASP) and agents that specifically prevent cellular senescence.

The compound may have more specific advantages, and consequent uses, in the fields indicated in the list below and, more preferably, to treat disorders, which may be pathologies or aesthetic disorders or others preferably associated with senescence, indicated below with a terminology English science clear to the skilled in any language.
Idiopathic pulmonary fibrosis (IPF), chronic obstructive pulmonary disease (COPD), asthma, cystic fibrosis, emphysema, bronchiectasis, and age-related loss of pulmonary function;
Chronic kidney disease (CKD), interstitial nephritis, glomerulosclerosis/glomerulonephritis, acute kidney disease (AKD), kidney failure;
Liver fibrosis, chronic hepatitis, non alcoholic fatty liver disease (NAFLD);
Pancreatic fibrosis, chronic pancreatitis;
Myocardial fibrosis, infarction;
Oral submucosa fibrosis;
Neurodegenerative Diseases, such as Alzheimer's, Parkinson's, Multiple Sclerosis, mild cognitive impairment, motor neuron dysfunction, Huntington's disease, dementia, etc.;
Neuropsychiatric disorders;
Toxicity or inflammation, induced by chemotherapies, radiotherapies, or any other medical procedure, such as for therapeutic, diagnostic, cosmetic purposes;
Acute and chronic viral diseases, such as HIV, Covid-19, etc.;
Osteoporosis, osteoarthritis, inflammatory bowel diseases (IBDs), inflammatory bowel syndrome (IBS), rheumatoid arthritis, oral mucositis, kyphosis, intervertebral disc degeneration, herniated intervertebral disc;
Adipose atrophy;
Sarcopenia, muscle/mobility loss due to ageing, muscle fatigue;
Atherosclerosis, angina, arrhythmia, cardiomyopathy, cardiomyocyte hypertrophy, congestive heart failure, coronary artery disease, carotid artery disease, endocarditis, coronary thrombosis, myocardial infarction, hypertension, aortic aneurysm, cardiac diastolic dysfunction, hypercholesterolemia, hyperlipidemia, mitral valve prolapsed, peripheral vascular disease, cardiac stress resistance, cardiac fibrosis, brain aneurysm, and stroke;
Rare Diseases associated with ageing and senescence, such as: aplastic anaemia, dyskeratosis congenita, Revetz syndrome, Hoyeraal-Hreidarsson syndrome, Lewy body dementia (LBD), amyloidosis, Paget's disease, diffuse idiopathic skeletal hyperostosis (DISH), multiple system atrophy (MSA), etc.;
Diabetes (Type 2, Type 1), diabetic ulcer, obesity, metabolic syndrome;
Wound healing;
Frailty;
Glaucoma, macular degeneration, cataracts, presbyopia, and vision loss;
Hearing Loss;
Immune function decline due to ageing (Immunosenescence);
Alopecia, Hair Loss;
Melasma, discoloured skin, eczema, psoriasis, hyperpigmentation, nevi, rashes, atopic dermatitis, urticaria, diseases and disorders related to photosensitivity or photoaging, rhytides, pruritis, dysesthesia, eczematous eruptions, eosinophilic dermatosis, reactive neutrophilic dermatosis, pemphigus, pemphigoid, immunobullous dermatosis, fibrohistocytic proliferations of skin, cutaneous lymphomas, and cutaneous lupus;
Diseases or pathological alterations or perfusion conditions associated to transplant of kidney, liver, lung, heart, pancreas or other organ, as well as of stem cells or other cells.

The said compound can be used alone or, in combination with other known drugs of the type selected from: senolytics, senomorphic, senostatic, senotherapeutic, cellular senescence promoters, and compounds which preserve the integrity of the tissues.

The compound according to the invention is preferably a synthetic derivative of the flavones, according to the formula (I) as indicated below.

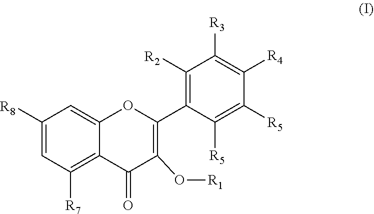

in which at least two of R₂-R₈₈ are H, and the remainder are individually selected from: H, OH, R₁, OR₁, NO₂, NH₂, NHR₁, F, Cl, Br, I, and more preferably R₂R₃R₆ are H and R₄R₅R₇ and R₈ are OH,
where R₁ is a radical and preferably selected from:
   H;
   C₁₋₂₄ alkyl or hetero-alkyl, C₁₋₂₄ alkenyl or hetero-alkenyl; C₁₋₂₄ alkynyl or hetero-alkynyl
   acyl residue of a fatty acid. Fatty acid which can be saturated, unsaturated, polyunsaturated, of both synthetic and natural origin.
More preferably R₁ is:

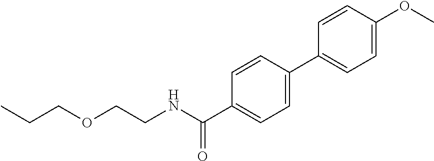 [SMILES: CCCOCCNC(=O)c1ccc(-c2ccc(OC)cc2)cc1]

### Alternatively, R₁ is:

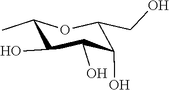

Alternatively still, R₁ is:

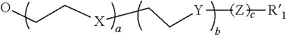

Where R₁ is chosen among the same compounds among which R₁ is chosen₁, where a, b, c are each between 0 and 12,
where X, Y and Z are each individually chosen from: CH2, O, N (R₁), S, NH, SO, SO₂, OC(O), CO, NHC(O), C(O) NH, NH—C(O)—NH, NH—C(S)—NH.
R₁ can be a Linker L and said linker is chosen from the following compounds:

| LINKER L | |
|---|---|
| L1 | 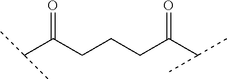 |
| L2 | 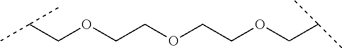 |
| L3 | 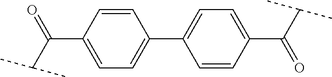 |
| L4 | 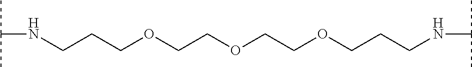 |
| L5 | 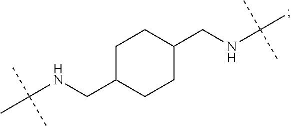 |
| | 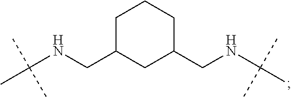 |
| | 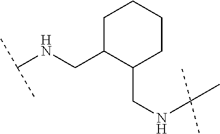 |
| L6 | 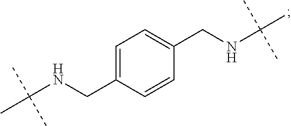 |
| | 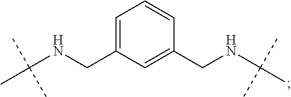 |
| | 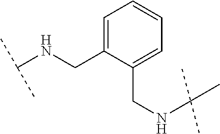 |
| L7 | 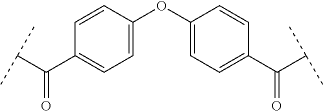 |
| L8 | 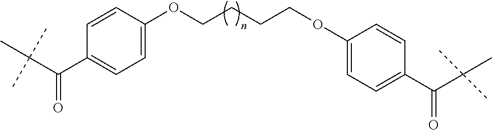 |
| | n = 0-8 |
| L9 | 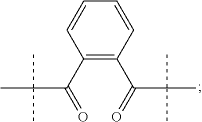 |
| | 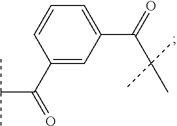 |
| | 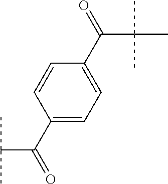 |
| L11 | 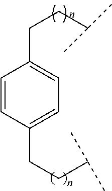 |
| | n = 1-6, each independently |
| L12 | 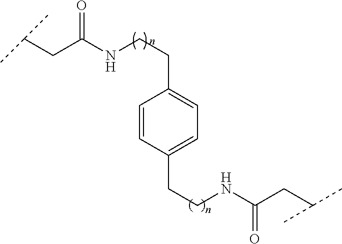 |
| | n = 1-6 |
| L13 | 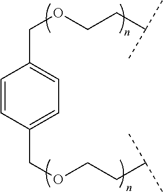 |
| | n = 1-3, each independently |
| L14 | 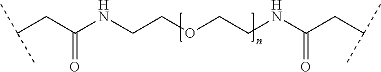 |
| | n = 1-4 |
| L15 | 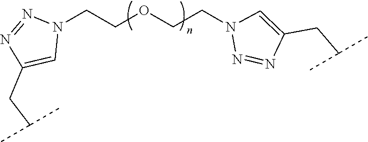 |
| | n = 1-4, each independently |
| L16 | 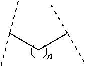 |
| | n = 2-12 |
| L17 | 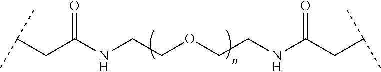 |
| | n = 1-5 |
| L18 | 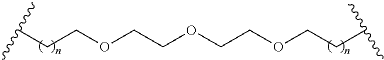 |
| | n = 1-3 |
| L19 | 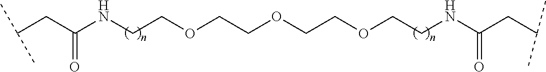 |
| | n = 1-3 |
| L20 | 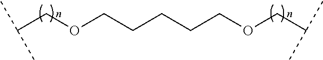 |
| | n = 1-3 |
| L21 | 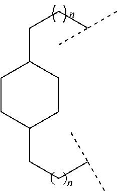 |
| | n = 1-6 |
| L22 | 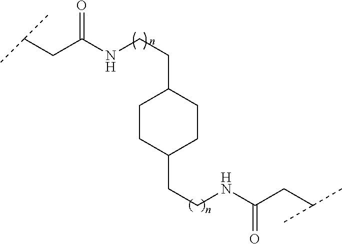 |
| | n = 1-6 |
| L23 | 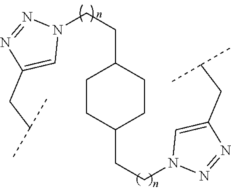 |
| | n = 1-6, each independently |
| L24 | 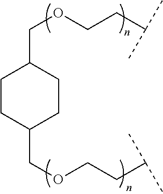 |
| | n = 1-3, each independently |
| L25 | 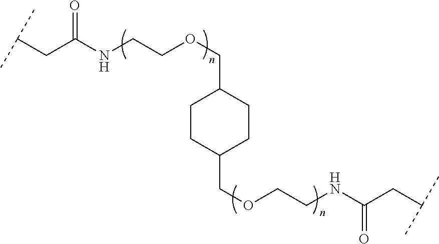 |
| | n = 1-3, each independently |
| L26 | 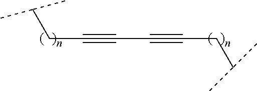 |
| | n = 1-6, each independently |
| L27 | 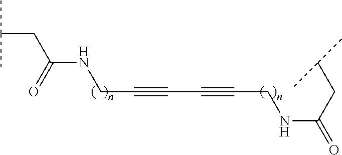 |
| | n = 1-6, each independently |
| L28 | 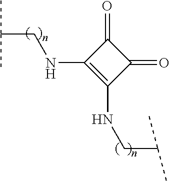 |
| | n = 1-6, each independently |
| L29 | 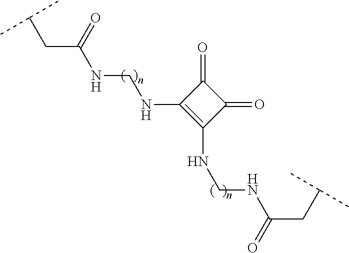 |
| | n = 1-6, each independently |
| L30 | 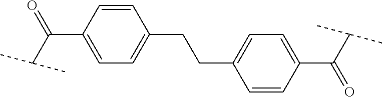 |
| L31 | 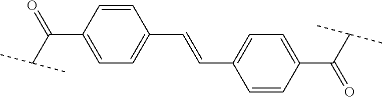 |
| L32 | 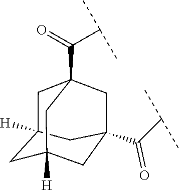 |
| L33 | 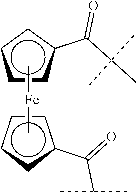 |
| L34 | 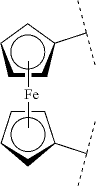 |
| L35 | 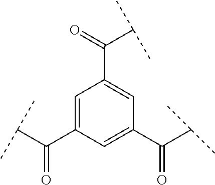 |
| L36 | 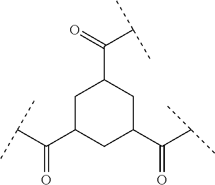 |
| L37 | 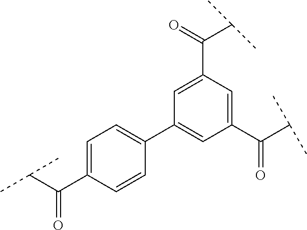 |
| L38 | 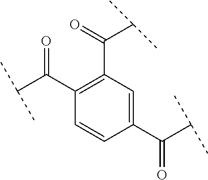 |
| L39 | 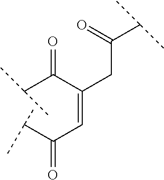 |
| L40 | 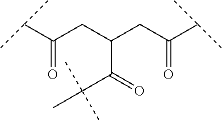 |
| L41 | 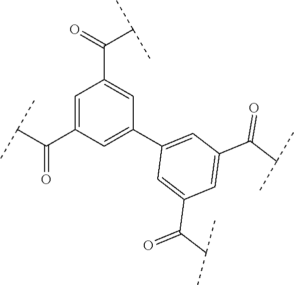 |
| L42 | 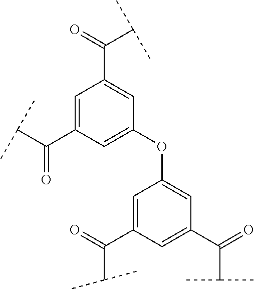 |
| L43 | 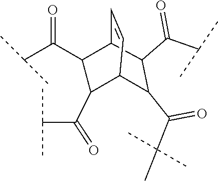 |
| L44 | 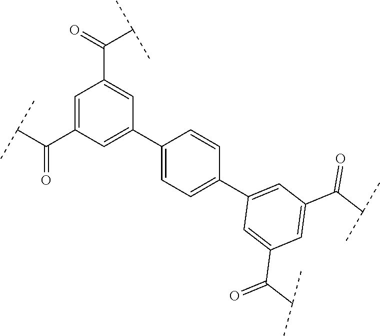 |
| L45 | 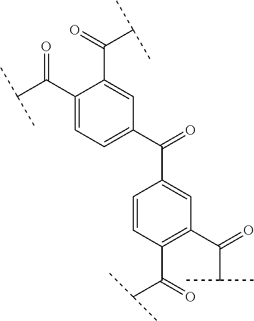 |
| L46 | 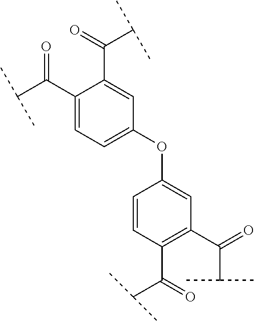 |
| L47 | 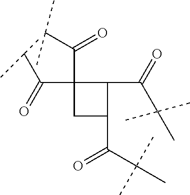 |
| L48 | 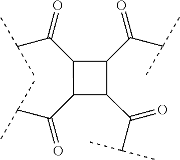 |
| L49 | 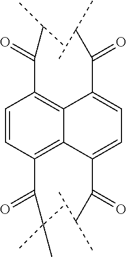 |
| L50 | 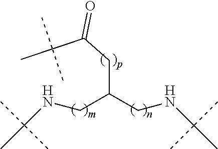 |
| | n, m, p = 0-6; each independently |
| L51 | 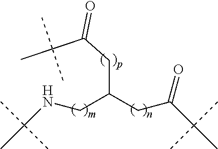 |
| | n, m, p = 0-6; each independently |
| L52 | 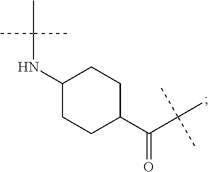 |
| | 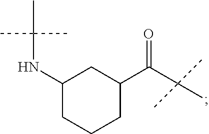 |
| | 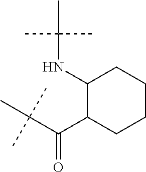 |
| L53 | 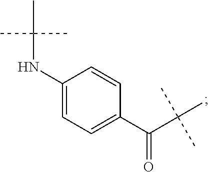 |
| | 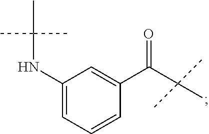 |
| | 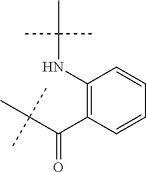 |
| L54 | 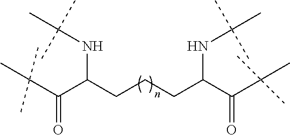 |
| | n = 0, 5 |
| L55 | 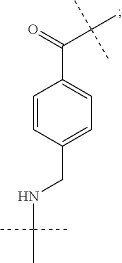 |
| | 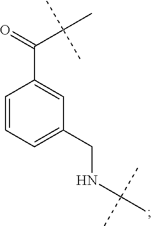 |
| | 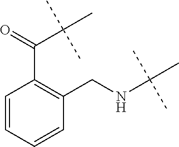 |
| L56 | 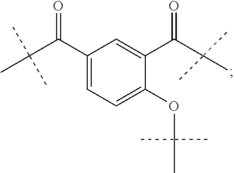 |
| | 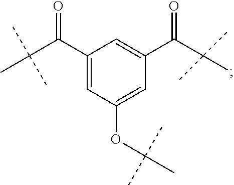 |
| | 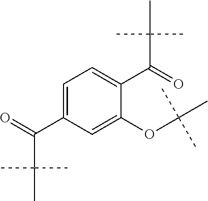 |
| L57 | 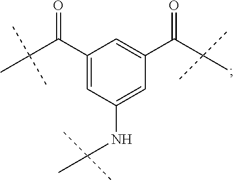 |
| | 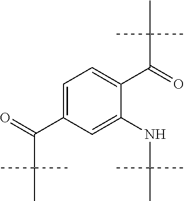 |
| L58 | 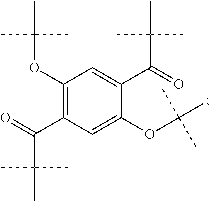 |
| | 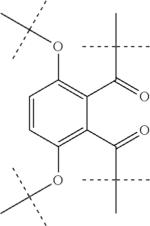 |
| L59 | 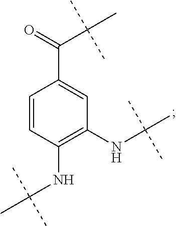 |
| | 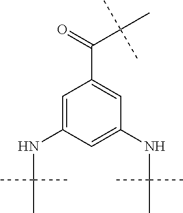 |
| L60 | 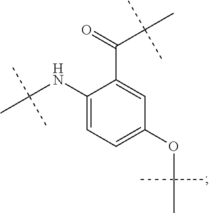 |
| | 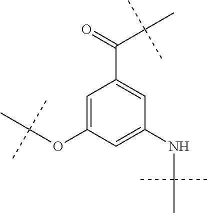 |
| L61 | 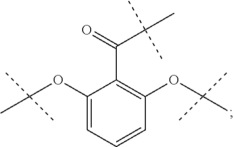 |
| | 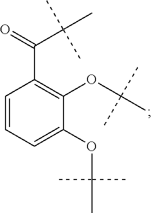 |
| | 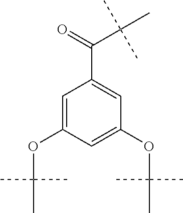 |
| L62 | 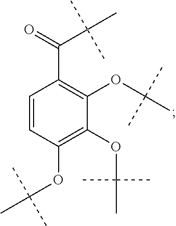 |
| | 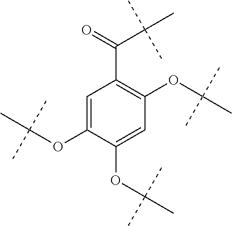 |
| L63 | 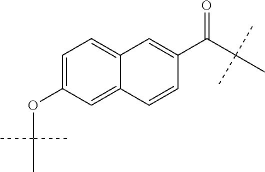 |
| L64 | 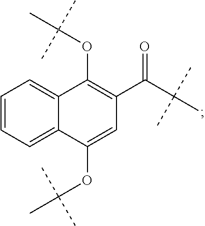 |
| | 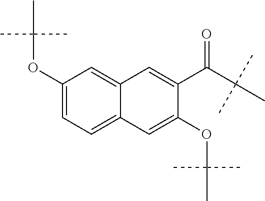 |
| L65 | 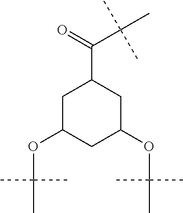 |
| L66 | 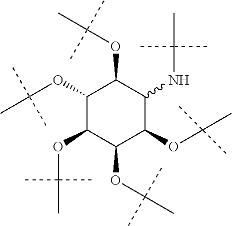 |
| L67 | 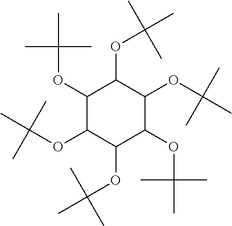 [SMILES: CC(C)(C)OC1C(OC(C)(C)C)C(OC(C)(C)C)C(OC(C)(C)C)C(OC(C)(C)C)C1OC(C)(C)C] |
| L68 | 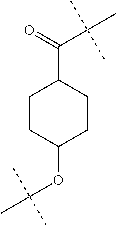 |
| L69 | 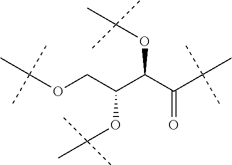 |
| L70 | 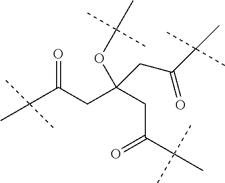 |
| L71 | 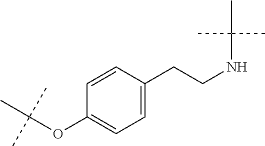 |
| L72 | 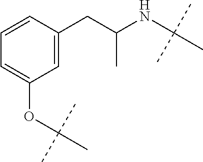 |
| L73 | 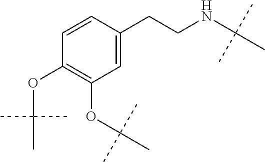 |
| L74 | 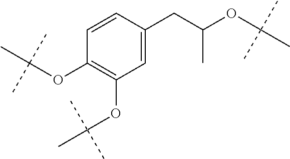 |
| L75 | 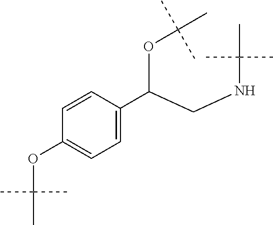 |
| L76 | 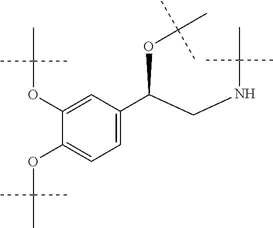 |
| L77 | 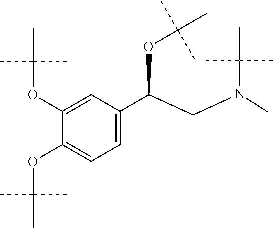 |
| L78 | 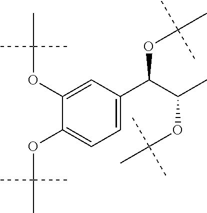 |
| L79 | 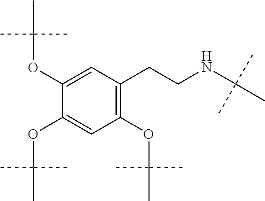 |
| L80 | 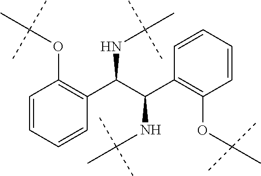 |
| L81 | 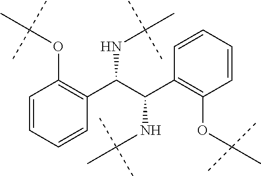 |
| L82 | 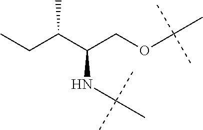 |
| L83 | 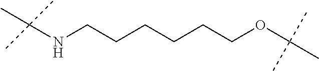 |
| L84 | 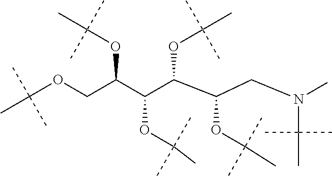 |
| L85 | 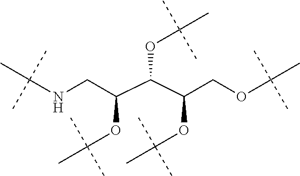 |
| L86 | 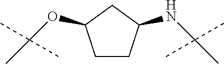 |
| L87 | 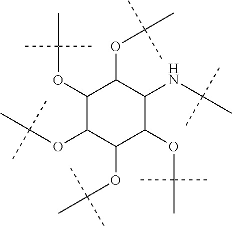 |
| L88 | 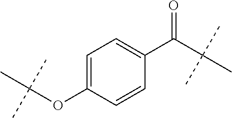 |
| L89 | 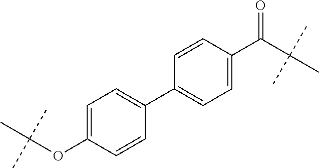 |
| L90 | 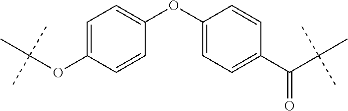 |

A preferred synthesis process of the compounds described is the following.

Generally, complex or hybrid molecules are synthesized in which a pharmacophoric appendage is linked, through a spacer bridge, to the oxygen in position 3 of quercetin, illustrated below, where the general structure of quercetins functionalized in position 3 with a generic pharmacophore is shown.

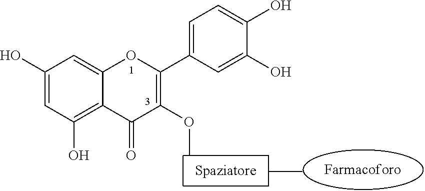

The synthesis of the biphenyl derivative, a compound according to the invention selected as a promising senotherapeutic, and in particular senolytic, in the experimental studies in vitro, originally prepared for the purpose of evaluate the importance of an aromatic portion in R₁.

Quercetin tetrabenzylated 7, in which the hydroxy in position 3 is free, is reacted with 2-[2-(Boc-amino) ethoxy] ethylbromide, the bromide of the ethereal spacer whose terminal amino function is protected by a butyloxy group carbonyl (boc). The compound 11b thus obtained is treated with HCl in THF/H₂O to remove the boc, and then debenzyled by catalytic hydrogenation with palladium on activated carbon (Pd—C) to give quercetin 27. This product represents a central point in the synthetic strategy, because thanks to its amino function it can be easily functionalized, for example with molecules that carry a carboxyl function, forming amides. Following this strategy, quercetin 27 is reacted with the diphenyl acid 28g previously activated with EDC·HCl and HOBt to obtain the desired amide (29g; Scheme 1).

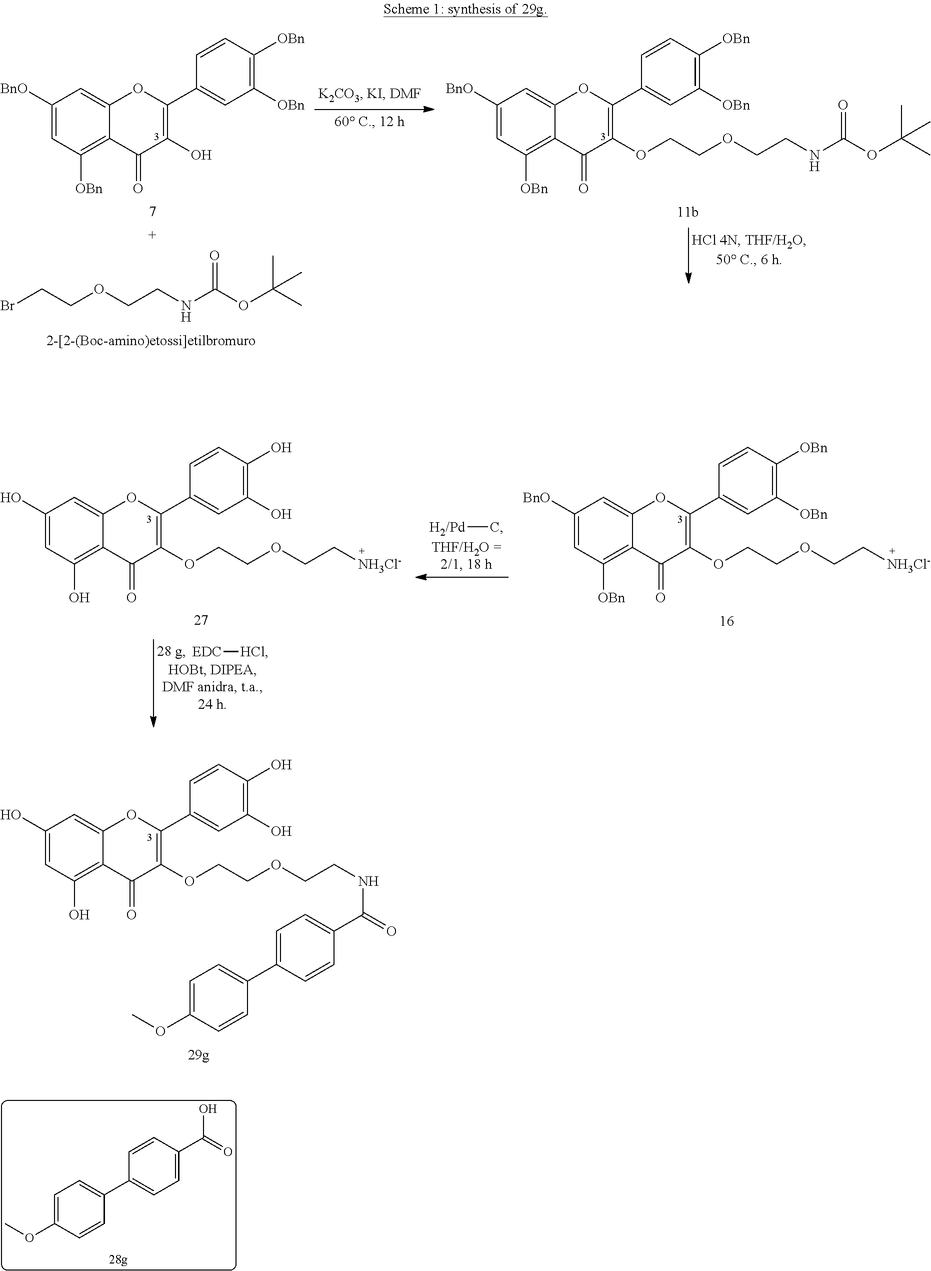

The compound 29g thus obtained is a senolytic consisting of a diphenyl system connected by an amino-ethereal spacer to position 3 of quercetin.

Regarding the materials and methods, all the chemical reagents used are of analytical quality and used as received.

The 1H and 13C-NMR experiments were recorded in deuterated solvents indicated in a Bruker Avance 400S™ spectrometer at 400.13 and 100.62 MHz respectively.

Coupling constants are reported in hertz and rounded to 0.1 Hz. Where required, chromatographic purifications were performed on silica gel for flash chromatography (70-230 mesh) using the specified eluents.

Where specified, reactions were performed using a CEM Discover microwave reactor.

The synthesis of 5,7-bis (benzyloxy)-2-(3,4-bis (benzyloxy) phenyl)-3-hydroxy-4H-chromen-4-one: 7, preferably takes place as described below.

To a solution of 2-(3,4-dihydroxyphenyl)-4,5-dihydroxy-3-[3,4,5-trihydroxy-6-[(3,4,5-trihydroxy-6-methyl-oxane-2-yl) oxymethyl] oxan-2-yl] oxy-cromen-7-one 6 (1 g; 1.6 mmol) in DMF (12 ml) anhydrous K2CO3 (1.81 g; 13.1 mmol) was added sequentially) and benzyl bromide (2.24 g; 13.1 mmoles). The reaction mixture is left under stirring in an argon atmosphere and at room temperature. On completion (approximately 24 hours), the reaction mixture was diluted with EtOAc (40 mL) and washed with H₂O (2×30 mL). The organic phase was dried with anhydrous Na2SO4, filtered and the solvent removed under reduced pressure. The residue is added with a mixture of HCl (37%)/MeOH=2/98 v/v, and heated under reflux (T°=65° C.) for 2 hours.

To complete, the mixture is allowed to cool down to room temperature, then filtered through a buchner funnel and finally, the orange solid is washed with cold methanol. The final product 7 thus obtained does not require a further final purification (450 mg; 42.45%).

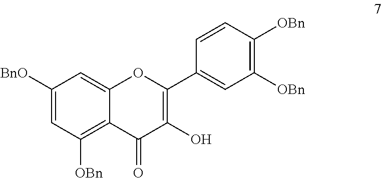 [SMILES: O=c1c(O)c(-c2ccc(OCc3ccccc3)c(OCc3ccccc3)c2)oc2cc(OCc3ccccc3)cc(OCc3ccccc3)c12]

1H NMR (400 MHz, CDCl3): 7.88 (s, 1H), 7.75 (d, 1H, J=6.5 Hz), 7.65-7.25 (m, 21H, Ph), 7.01 (d, 2H), 6.56 (s, 1H, J=8.7 Hz), 6.45 (s, 1H), 5.26 (s, 2H, CH2Ph), 5.23 (s, 2H, CH2Ph), 5.21 (s, 2H, CH2Ph), 5.11 (s, 2H, CH2Ph);

13C NMR (100 MHz, CDCl3): 171.70, 163.19, 159.33, 158.63, 150.14, 148.58, 141.83, 137.68, 137.13, 136.82, 136.15, 135.59, 128.74, 128.62, 128.53, 128.45, 127.89, 127.85, 127.77, 127.62, 127.53, 127.18, 126.63, 124.26, 121.20, 114.17, 106.68, 97.52, 93.66, 71.52, 70.93, 70.67, 70.53.

The synthesis of compound 11b preferably takes place as described below.

To a solution of 5,7-bis (benzyloxy)-2-(3,4-bis (benzyloxy) phenyl)-3-hydroxy-4H-cromen-4-one 7 (500 mg; 0.754 mmol) in anhydrous DMF (4 ml), 2-[2-(Boc-amino) ethoxy] ethylbromide (404 mg; 1.508 mmol), anhydrous K2CO3 (250 mg; 1.810 mmol) and KI (36 mg; 0.226 mmol) were sequentially added. The reaction mixture is left under stirring at a temperature of 65° C. for 12 hours. For completeness, the mixture was partitioned between H₂O (10 mL) and EtOAc (20 mL). The organic phase was washed with a saturated aqueous solution of NaCl and the aqueous phase extracted with EtOAc (20 ml). The combined organic phases were dried over anhydrous Na2SO4 and the solvent removed under reduced pressure. The residue, obtained as yellow oil, was purified by flash column chromatography (SiO2; EtOAc/n-hexane 25%). Product 11b was isolated as a white oil (460 mg; 72%).

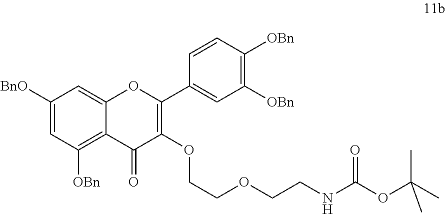 [SMILES: CC(C)(C)OC(=O)NCCOCCOc1c(-c2ccc(OCc3ccccc3)c(OCc3ccccc3)c2)oc2cc(OCc3ccccc3)cc(OCc3ccccc3)c2c1=O]

1H NMR (400 MHz, DMSO-d6): 7.83 (bs, 1H), 7.63 (d, 1H, J=4.0 Hz), 7.46-7.18 (m, 21H), 6.95 (d, 1H, J=2.0 Hz), 6.76 (bt, 1H, J=8.0 Hz), 6.70 (d, 1H, J=2.0 Hz), 5.25 (m, 8H), 4.12 (m, 2H), 3.60 (m, 2H), 3.32 (m, 2H), 3.03 (m, 2H), 1.35 (s, 9H).

13C NMR (100 MHz, DMSO-d6): 173.2, 163.6, 159.0, 156.5, 152.5, 151.2, 148.6, 140.3, 138.1, 137.8, 137.7, 137.0, 129.5, 129.4, 129.3, 129.2, 129.0, 128.9, 128.8, 128.6, 128.5, 127.8, 123.9, 123.3, 115.1, 114.6, 109.8, 98.7, 95.1, 78.6, 71.6, 71.4, 71.0, 70.9, 70.3, 70.0, 29.1.

The synthesis of compound 16 preferably takes place as described below.

An aqueous solution of 4N HCl (4 ml) was added to a solution of 11a (460 mg; 0.541 mmoles) in THF (6 ml). The reaction mixture is left under stirring at 50° C. for 6 hours. To complete, the reaction mixture was brought to dryness under reduced pressure and the residue, a white solid was used in the subsequent reaction without further chromatographic purification, 16 (quantitative).

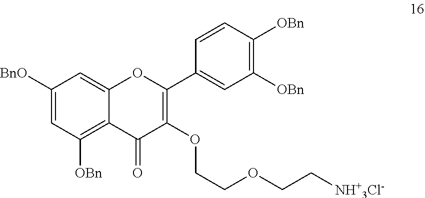

1H NMR (400 MHz, CD3OD): 7.66-7.30 (m, 22H), 7.05 (d, 1H, J=8.4 Hz), 6.57 (d, 1H, J=2.0 Hz), 6.51 (d, 1H, J=2.0 Hz), 5.27 (s, 2H), 5.25 (s, 2H), 5.17 (s, 2H), 5.12 (2H), 3.83-3.74 (m, 2H, 3.72-3.64 (m, 2H), 3.60-3.53 (m, 2H), 3.22-3.12 (m, 2H).

The synthesis of compound 27 preferably takes place as described below.

A solution of 16 (0.01 mmol) in a mixture of EtOH/THF (1:2% v/v) The mixture was added with Pd/C catalyst (10%) The reaction mixture was subjected three times to a vacuum
nitrogen cycle to remove both air from the system; done one last time the vacuum, the flask was filled with hydrogen up to a pressure of 1.2 Bar. Finally, the reaction mixture is left under stirring at room temperature until complete debenzylation of the desired product (about 12 hours). To complete, the catalyst is removed from the reaction mixture by filtration, and the solvent removed a reduced pressure and the residue was washed with a mixture of n-hexane/Et2O 70:30 (3×10 ml).

Product 27 was isolated as a yellow amorphous solid (74 mg; 95.0%).

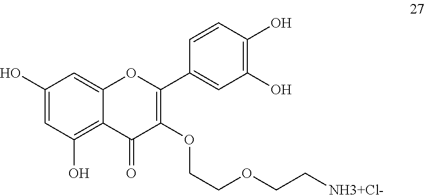

1H NMR (400 MHz, CD3OD): 7.67 (d, 1H, J=2.0 Hz), 7.52 (dd, 1H, J1=8.4 Hz, J2=2.0 Hz), 6.39 (d, 1H, J=1.6 Hz), 6.19 (d, 1H, J=2.0 Hz), 4.10-4.01 (m, 2H), 3.83-3.75 (2H, m), 3.74-3.67 (2H, m), 3.19-3.10 (2H, m); 13C NMR (100 MHz, CD30D): 179.9, 166.0, 163.0, 158.5, 158.4, 150.0, 146.3, 138.3, 122.8, 122.5, 117.0, 116.4, 105.8, 99.9, 94.8, 72.9, 71.2, 67.8, 40.6.

The synthesis of compound 29g preferably takes place as described below.

EDC·HCl (19.8 mg; 0.10 mmol) was added to a solution of the acid 28g (15.8 mg; 0.069 mmol) in anhydrous DMF (2 ml), under an argon atmosphere and with magnetic stirring. and after 30 minutes HOBt (11.2 mg; 0.083 mmol). After a further 30 minutes, the solution obtained was added drop by drop, in 5 minutes using a syringe, to a solution of compound 27 (32 mg; 0.063 mmol) and DIPEA (100 μl; 0.57 mmol) in anhydrous DMF (3 ml), under stirring, maintained at 0° C. and under an argon atmosphere.

The reaction thus obtained is brought to room temperature and maintained under stirring and argon atmosphere for 24 hours, after which the reaction was diluted with EtOAc (25 ml) and extracted with water (2 times) and a saturated solution of NaCl (1 time). The organic phase was dried over anhydrous Na2SO4, filtered and concentrated in vacuo to dryness to give a crude which was purified by flash column chromatography (SiO2; DCM/MeOH=2->4%) to give the compound 29g as a yellow solid (9 mg; 22%).

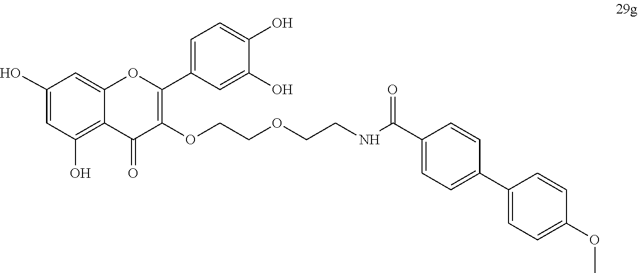 [SMILES: COc1ccc(-c2ccc(C(=O)NCCOCCOc3c(-c4ccc(O)c(O)c4)oc4cc(O)cc(O)c4c3=O)cc2)cc1]

1H NMR (400 MHz, CD3OD): 7.82-7.77 (m, 2H), 7.68 (d, 1H, J=2.4 Hz), 7.59-7.47 (m, 5H), 6.99-6.93 (m, 2H), 6.89 (d, 1H, J=8.4 Hz), 6.34 (d, 1H, J=1.6 Hz), 6.16 (d, 1H, J=1.6 Hz), 4.15-4.09 (m, 2H), 3.81 (s, 3H), 3.79-3.73 (m, 2H), 3.66-3.61 (m, 2H), 3.61-3.55 (m, 2H);

13C NMR (100 MHz, CD3OD): 179.9, 170.3, 165.8, 163.0, 161.2, 158.3, 158.0, 149.9, 146.3, 145.2, 138.3, 133.52, 133.48, 129.1, 128.8, 127.3, 123.0, 122.6, 117.0, 116.3, 115.3, 105.8, 99.8, 94.7, 72.8, 71.3, 70.6, 55.7, 41.1.

The invention also relates to the synthesis of ethers 1, 2 and 3 of quercetin, the synthetic strategy of which provides for an orthogonal protection of quercetin as reported by Rolando and coll. (Tetrahedron Letters, 2011, 52, 4738). The synthesis of ethers 1, 2 and 3 of quercetin, starting from commercial routine 6, involves an exhaustive benzylation of the free hydroxyl groups, followed by the selective hydrolysis of the disaccharide in position 3 and consequent alkylation with the appropriate alkyl-bromide and finally, removal of benzyl groups by hydrogenation catalyzed by Pd/C. The strategy is simplified below:

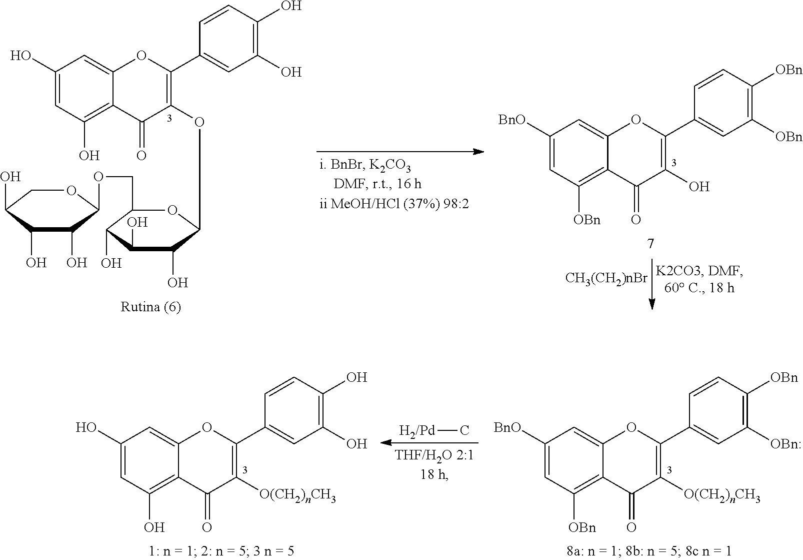

Furthermore, the invention also relates to the synthesis of esters 4 and 5 of quercetin, carried out by means of the enzymatic strategy reported by D. Lambusta and coll. (J. Mol. Catal. B-Enzymatic, 2003, 22, 271). The strategy involves the esterification of quercetin 9 on all five hydroxyl groups and consequent selective alcoholysis mediated by the appropriate choice of Candida antarctica (CAL) and Mucor miehei (MML) lipases, as shown in the following scheme.

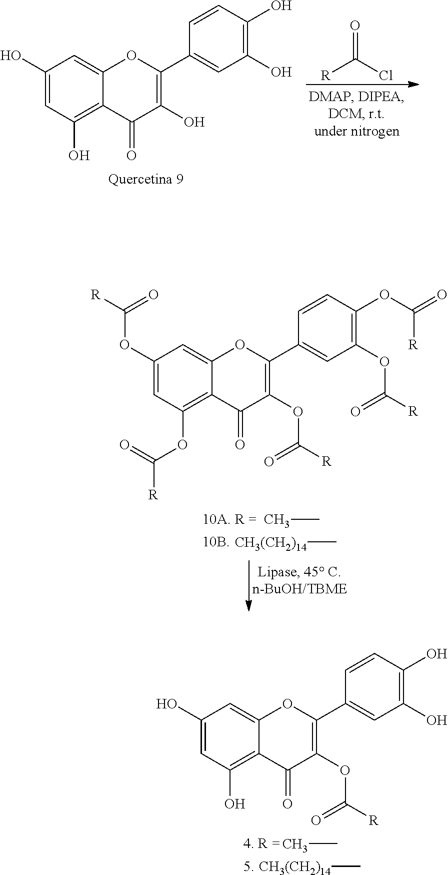

Similarly to what has been described for the synthesis of products 8, below we report the synthesis of the products of the 11 series, as shown in the following scheme.

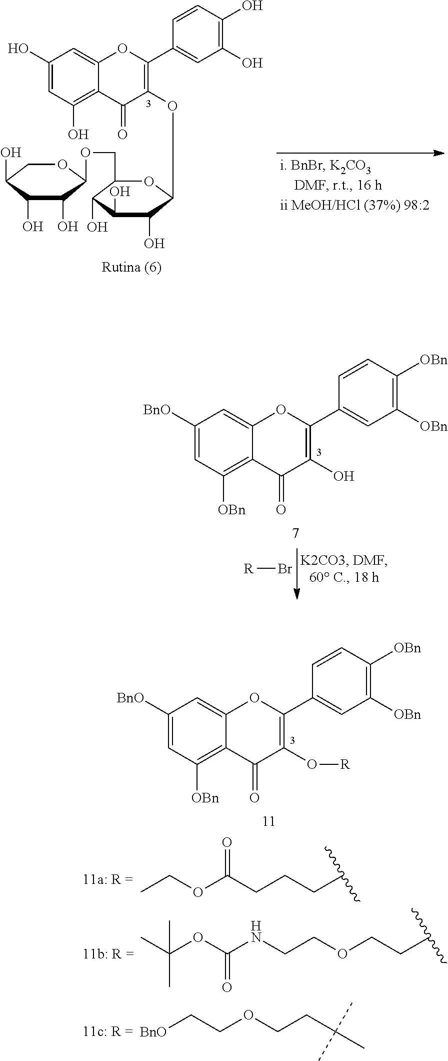

The compounds according to the invention also include di-, tri- and tetra-meric molecules, where two or more quercetin units are linked, through position 3 and a spacer to a suitable central linker (scaffold), as follows reported.

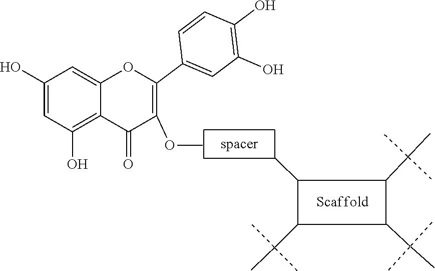

The choice of both the spacer and the central scaffold was made by trying to study the effect due to the flexibility and distance between the quercetin units.

A first group of dimeric molecules was synthesized by the strategy reported in the following scheme, where the ethyl ester of intermediate 11a is hydrolyzed by treatment with sodium hydroxide in THF/H₂O and the corresponding carboxylic acid 12, activated with EDC/HOBt, it is condensed with the suitable diamine (H2N-Linker-NH2) 13. Finally, the final products 15 were obtained by exhaustive debenzylation of the intermediates 14 carried out by classical catalytic hydrogenation.

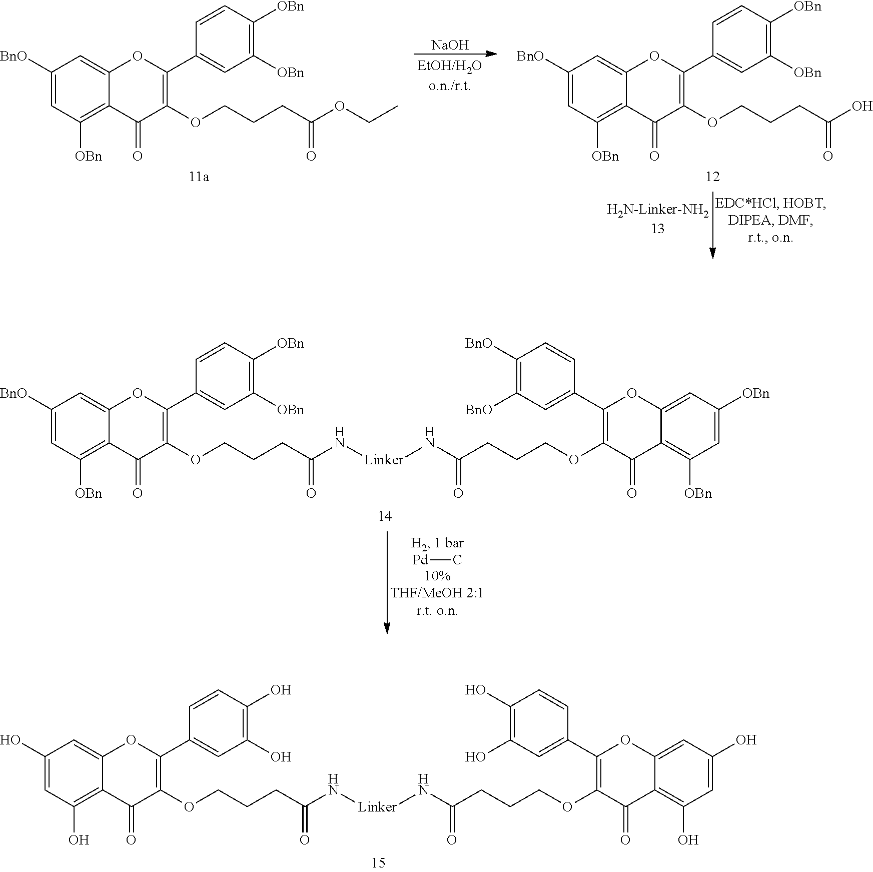

The compound according to the invention can also be made by means of one or more processes described in patent application WO 2019/008537 A1, filed on May 7, 2017 in the name of Vera Salus Ricerca SrI from page 8 line 10 to page 90 last line. Such methods and molecules are also considered included by reference in the present document, contribute to solving secondary technical problems and are therefore part of the invention.

The said compound can also be produced by means of processes described in patent application WO-A-99/66062 of the National Research Council and of Rao-Erbe of Rao Felice, from page. 3 line 8 on page 9 line 22, which are incorporated herein by reference. However, other similar methods can be used.

The invention therefore also defines a new process for eliminating senescent cells, or for solving problems associated with senescence by taking the substances described and a new process for making drugs or compounds or substances for treating the aforementioned problems.

According to the applicant's experiments, the compounds according to the invention have been shown to possess senotherapeutic activity, and in particular senolytic activity, in different cellular models, corresponding to human tissues of various origins. Human cell senescence was induced in vitro by treatment with doxorubicin (for 24 h), at a concentration that does not induce apoptosis (100-200 nM), and subsequent cell recovery (in optimal culture medium, without treatments) to 10-12 days. The successful conversion to the senescent cell phenotype was confirmed by morphological changes (flattening and cell enlargement) and positivity for lysosomal beta-galactosidase (β-GAL). The SA-β-GAL (senescence-associated β-GAL) senescence assay was performed with the SA-β-Gal Staining Kit (Cell Signaling Technology, Inc., Danvers, MA). Briefly, after fixation with 20% formaldehyde for 15 min at room temperature, senescent cells were quantified by calculating the percentage of SA-β-GAL positive (blue stained) cells present in culture by examining 200 cells per well with the phase contrast microscope EVOS XL Cell Imaging System (Thermo Fisher; objective, 40X).

Senolytic activity on senescent human cells (in 96-well plates) was evaluated after specific treatments (for 72 h), including vehicle (DMSO; negative control), quercetin (Que; positive control) or different compounds according to the ‘invention bearing identification codes as fully illustrated also in patent application WO 2019/008537 A1, filed on May 7, 2017 in the name of Vera Salus Ricerca SrI (FIGS. 1, 2 and 3) from page 91 to page 106 and in the relative Figures. At the end of the treatments, cell survival was quantified by crystal violet staining. Briefly, cells were fixed with paraformaldehyde (4%) in PBS for 20 min, washed (2X in distilled water) and stained with 1% crystal violet (for 20 min). After further washing with water (3X), 100 μl of acetic acid (10%) were added per well and the absorbance (λ=590 nm) measured with a Synergy spectrophotometer (AHSI). Each experiment was carried out in quadruplicate and repeated at least three times, on three different days. Senolytic activity was expressed as % of senescent cells eliminated with respect to the control condition (DMSO). Results were presented as means±SEM (standard error of mean) and related analyzes were performed with GraphPadPrism® 6.0 software (CA, USA).

In a first step, the senolytic activities of the compounds according to the invention were investigated in human WS1 fibroblast cells (normal fibroblasts of cutaneous origin, ATCC® CRL-1502™). WS1 cell senescence was induced with 100 nM doxorubicin (24 h) and 12 days of recovery, while the senolytic activity was quantified after treatments with vehicle (DMSO), quercetin (Que, 20 μM), quercetin (20 μM) plus dasatinib (DAS, 200 nM; other positive control) or 18 distinct compounds according to the invention used at a concentration of 20 μM, alone (FIG. 1a) or in the presence of 200 nM dasatinib (FIG. 1b).

Almost all the compounds examined according to the invention exhibited inhibitory activity on WS1 human senescent cells (FIGS. 1a, 1b). When compared to quercetin or the combination quercetin plus dasatinib (known human selective senolytics), the majority of the compounds according to the invention have been shown to have a similar or superior senolytic efficacy (FIGS. 1a, 1b). Compared to quercetin, in fact, almost all of the compounds tested according to the invention exhibit higher inhibitory activity, with senolytic efficacy in many cases more than 2 times higher (FIG. 1a). Compared to the combination quercetin plus dasatinib, however, many of the compounds examined exert a similar senolytic action on senescent WS1 fibroblasts, and a compound (29g, indicated in the figure as VSR-017) is more effective (FIG. 1b). Of note, the compound according to the invention 29g, used alone, showed a higher senolytic effect than all the other compounds, both alone (FIG. 1a) and in combination with dasatinib (FIG. 1b). In particular, the senolytic effect of 29g (˜60%) was ˜3 times greater than that of quercetin (FIG. 1a) and also greater than the combination of quercetin plus dasatinib (FIG. 1b).

Subsequently, the senolytic activities of the compounds according to the invention were examined in other cellular models, including human intestinal epithelium cells HCT116 (colorectal adenocarcinoma cells, ATCC® CCL-247), human renal epithelial cells 786-0 (renal adenocarcinoma cells, ATCC® CRL-1932), human pancreatic epithelial cells PANC-1 (pancreatic cancer cells, ATCC® CRL-1469), human hepatic epithelial cells HepG2 (hepatocellular carcinoma cells, ATCC® HB-8065) and human lung epithelium cells A549 (lung adenocarcinoma cells, ATCC® CCL-185). In these cells, senescence was induced with 200 nM doxorubicin (24 h) and 10 (for HCT116 and A549) or 12 (for 786-0, PANC-1 and HepG2) days of recovery, while senolytic activity was quantified after treatments with vehicle (DMSO) or scalar concentrations (range, 1-60 μM) of quercetin (Que; positive control) or of each of the investigated compounds according to the invention (FIGS. 2a-3c).

All the compounds according to the invention exhibited senolytic activity on each of the 5 tissue types of human senescent cells examined, with effects significantly higher than quercetin induced by specific compounds at concentrations 10 μM (FIGS. 2a-3c). In this context, the following compounds according to the invention (belonging to distinct chemical classes) showed senolytic activities significantly higher than quercetin in the following human cell lines: compounds 29g, 15c and 19b in the senescent intestinal cells HCT116 (FIG. 2a); compounds 29g and 15c in senescent kidney cells 786-0 (FIG. 2b) and in senescent pancreatic cells PANC-1 (FIG. 3a); compounds 29g and 3 in senescent liver cells HepG2 (FIG. 3b), and compounds 29g, 15c, 3 and 31 in senescent lung cells A549 (FIG. 3c). Of note, the compound according to the invention 29g showed maximum senolytic effects in all the cell lines examined (FIGS. 1a-3c), indicating specificity and superior senolytic potency possessed by this compound compared to all the other treatments examined, regardless of the tissue. origin of senescent cells (pansenolytic activity).

Compound 15c also proved to be an effective pansenolytic (FIGS. 1a-3c), with the exception of senescent liver cells (FIG. 3b). Furthermore, some compounds according to the invention have been shown to possess selective senolytic effects, characterized by inhibitory potencies on higher senescent cells in certain tissue contexts, such as compound 19b in intestinal cells (FIG. 2, graph on the left), compound 31 in cells pulmonary (FIG. 3c) and compound 3 in liver (FIG. 3b) and lung cells (FIG. 3c).

These results demonstrate that the compounds according to the invention show very promising specific, elevated and/or selective senolytic activities, indicating that they could be developed as new senotherapeutic drugs and in specific applications for the prevention or treatment of human pathologies associated with aging.

These results therefore demonstrate that the compounds according to the invention exhibit a specific, important and very promising senotherapeutic, and in particular senolytic, activity, indicating that they could be developed as new senotherapeutic drugs for humans.

1. A senotherapeutic compound comprising a synthetic derivative of flavones, according to the formula (I): 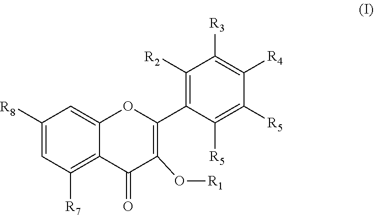
wherein at least two of R₂-R₈ are H, and the remaining are individually chosen from:
H, OH, R₁, OR₁, NO₂, NH₂, NHR₁, F, Cl, Br, I,
where R1 is a radical.

2. The senotherapeutic compound according to claim 1, wherein R₁ is selected from:
H;
C₁₋₂₄ alkyl or hetero-alkyl, C₁₋₂₄ alkenyl or hetero-alkenyl; C₁₋₂₄ alkynyl or hetero-alkynyl;
acyl residue of a fatty acid.

3. The senotherapeutic compound according to claim 1, wherein:
R₄, R₅, R₇ and R₈ are OH,
R₂, R₃, R₆, are H.

4. The senotherapeutic compound according to claim 1, wherein R₁ is:

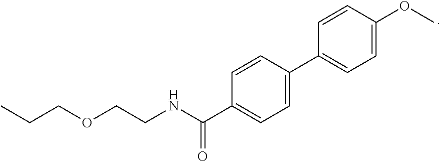 [SMILES: CCCOCCNC(=O)c1ccc(-c2ccc(OC)cc2)cc1]

5. The senotherapeutic compound according to claim 1, wherein R₁ is:

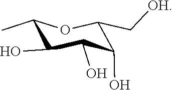

6. The senotherapeutic compound according to claim 1, wherein R₁ is: 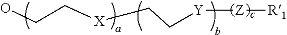
where R′₁ is chosen from:
H;
C₁₋₂₄ alkyl or hetero-alkyl, C₁₋₂₄ alkenyl or hetero-alkenyl; C₁₋₂₄ alkynyl or hetero-alkynyl
acyl residue of a fatty acid,
a linker L,
where a, b, c are each between 0 and 12,
where X, Y and Z are each individually chosen from: CH2, O, N(R1), S, NH, SO, SO2, OC(O), CO, NHC(O), C(O)NH, NH—C(O)—NH, NH—C(S)—NH.

7. The senotherapeutic compound according to claim 6, wherein R′₁ is a Linker L and said linker is selected from the following compounds:

| LINKER L | |
|---|---|
| L1 | 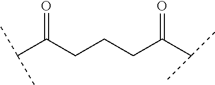 |
| L2 | 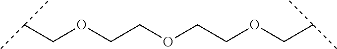 |
| L3 | 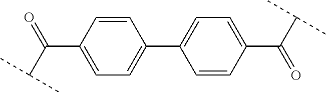 |
| L4 | 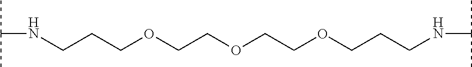 |
| L5 | 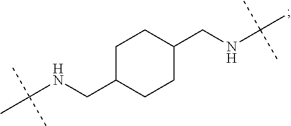 |
| | 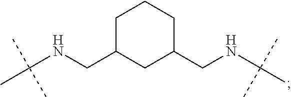 |
| | 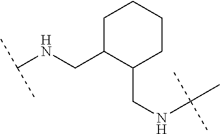 |
| L6 | 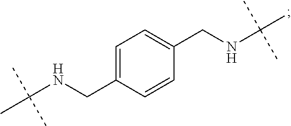 |
| | 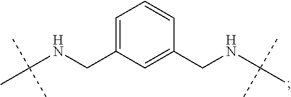 |
| | 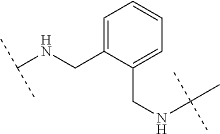 |
| L7 | 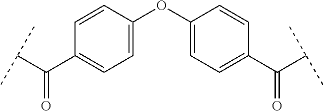 |
| L8 | 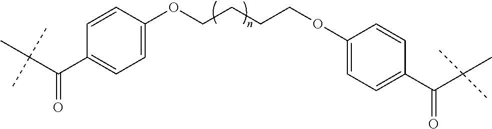 |
| | n = 0-8 |
| L9 | 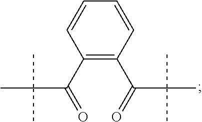 |
| | 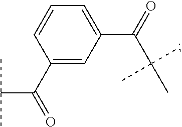 |
| | 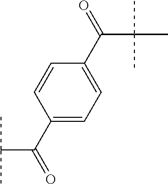 |
| L11 | 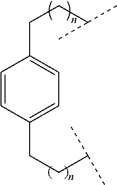 |
| | n = 1-6, each independently |
| L12 | 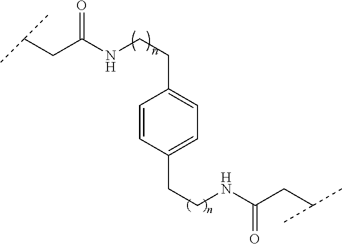 |
| | n = 1-6 |
| L13 | 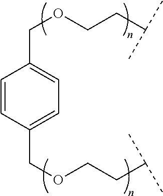 |
| | n = 1-3, each independently |
| L14 | 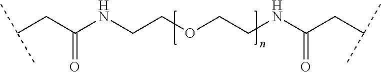 |
| | n = 1-4 |
| L15 | 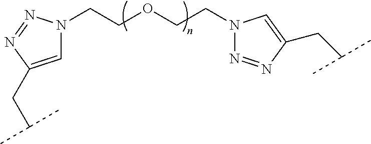 |
| | n = 1-4, each independently |
| L16 | 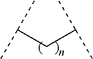 |
| | n = 2-12 |
| L17 | 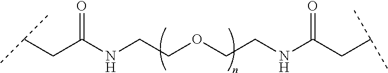 |
| | n = 1-5 |
| L18 | 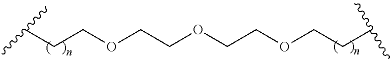 |
| | n = 1-3 |
| L19 | 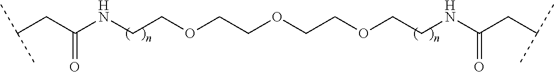 |
| | n = 1-3 |
| L20 | 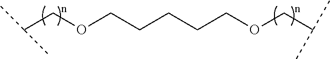 |
| | n = 1-3 |
| L21 | 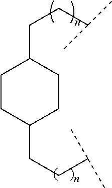 |
| | n = 1-6 |
| L22 | 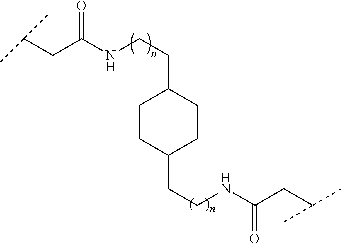 |
| | n = 1-6 |
| L23 | 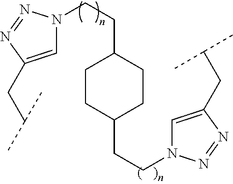 |
| | n = 1-6, each independently |
| L24 | 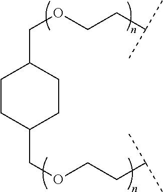 |
| | n = 1-3, each independently |
| L25 | 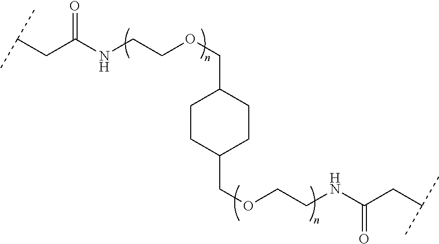 |
| | n = 1-3, each independently |
| L26 | 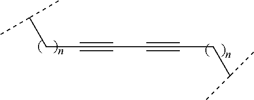 |
| | n = 1-6, each independently |
| L27 | 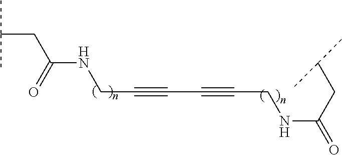 |
| | n = 1-6, each independently |
| L28 | 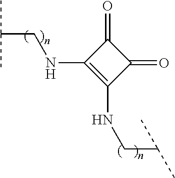 |
| | n = 1-6, each independently |
| L29 | 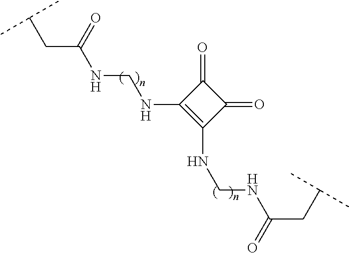 |
| | n = 1-6, each independently |
| L30 | 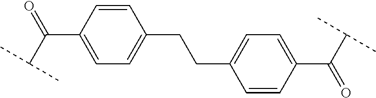 |
| L31 | 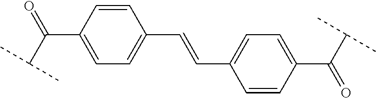 |
| L32 | 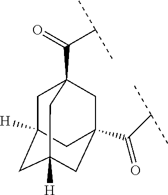 |
| L33 | 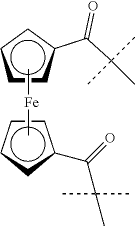 |
| L34 | 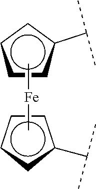 |
| L35 | 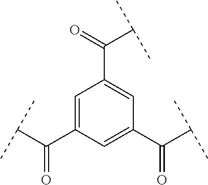 |
| L36 | 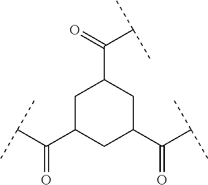 |
| L37 | 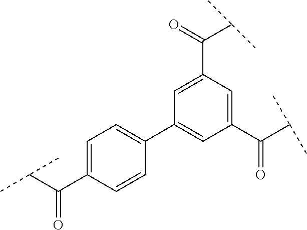 |
| L38 | 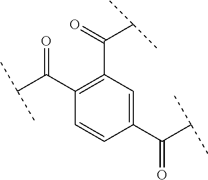 |
| L39 | 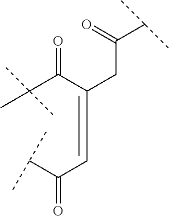 |
| L40 | 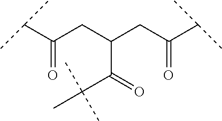 |
| L41 | 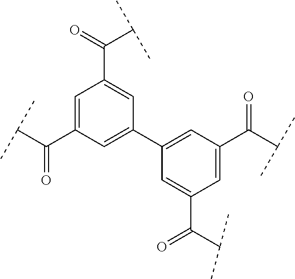 |
| L42 | 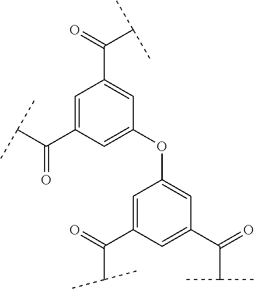 |
| L43 | 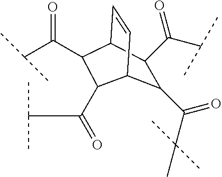 |
| L44 | 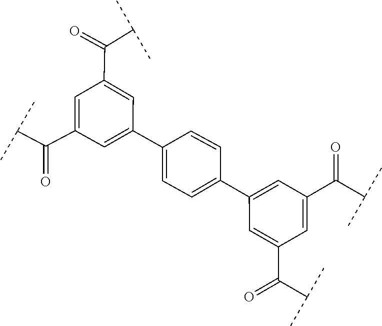 |
| L45 | 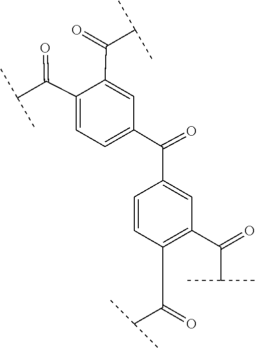 |
| L46 | 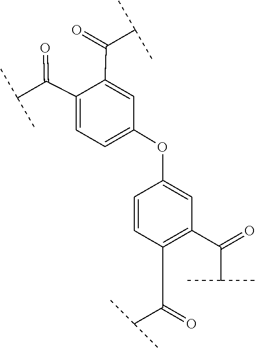 |
| L47 | 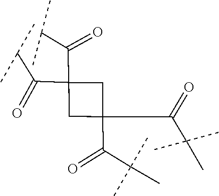 |
| L48 | 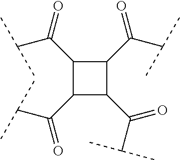 |
| L49 | 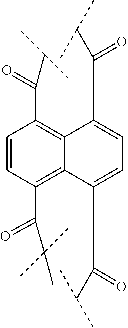 |
| L50 | 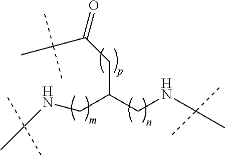 |
| | n, m, p = 0-6; each independently |
| L51 | 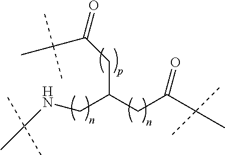 |
| | n, m, p = 0-6; each independently |
| L52 | 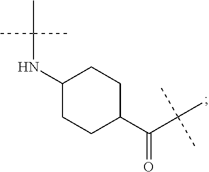 |
| | 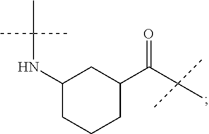 |
| | 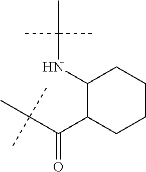 |
| L53 | 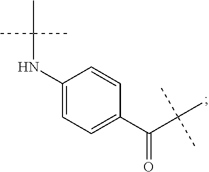 |
| | 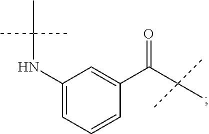 |
| | 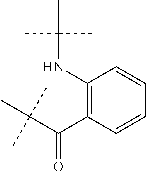 |
| L54 | 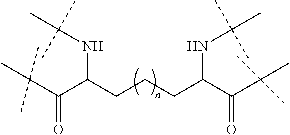 |
| | n = 0, 5 |
| L55 | 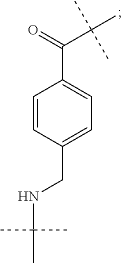 |
| | 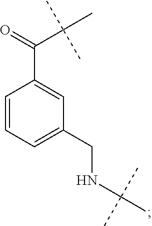 |
| | 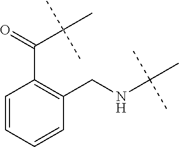 |
| L56 | 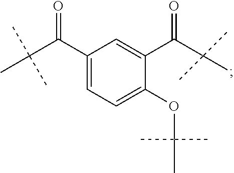 |
| | 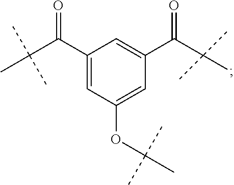 |
| | 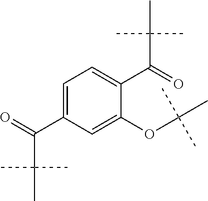 |
| L57 | 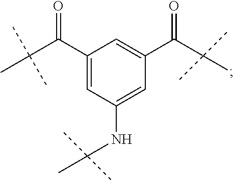 |
| | 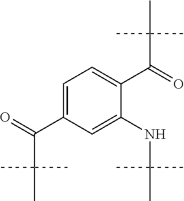 |
| L58 | 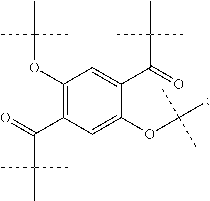 |
| | 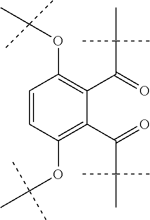 |
| L59 | 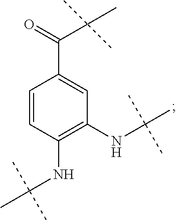 |
| | 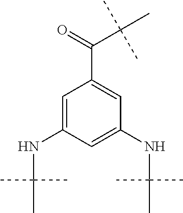 |
| L60 | 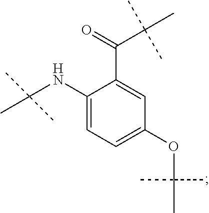 |
| | 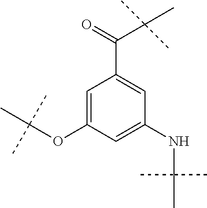 |
| L61 | 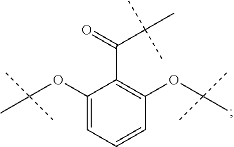 |
| | 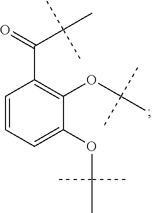 |
| | 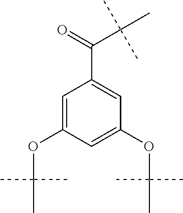 |
| L62 | 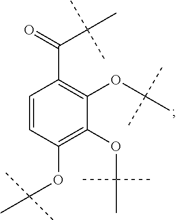 |
| | 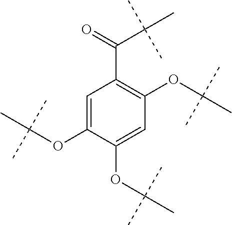 |
| L63 | 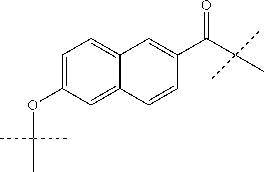 |
| L64 | 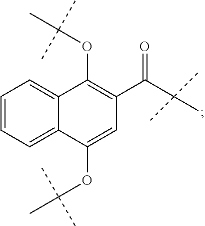 |
| | 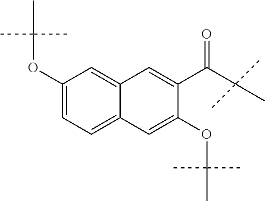 |
| L65 | 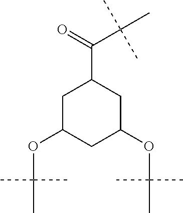 |
| L66 | 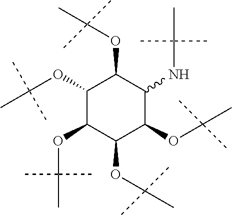 |
| L67 | 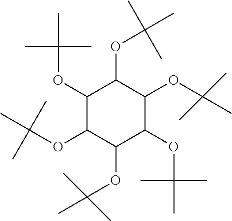 [SMILES: CC(C)(C)OC1C(OC(C)(C)C)C(OC(C)(C)C)C(OC(C)(C)C)C(OC(C)(C)C)C1OC(C)(C)C] |
| L68 | 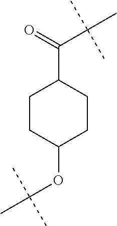 |
| L69 | 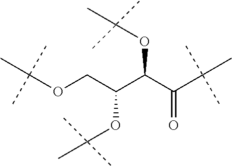 |
| L70 | 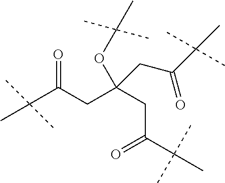 |
| L71 | 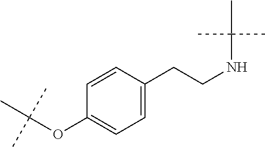 |
| L72 | 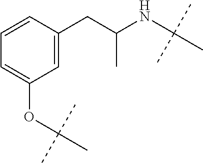 |
| L73 | 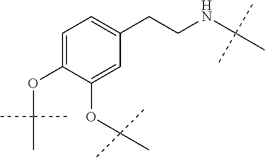 |
| L74 | 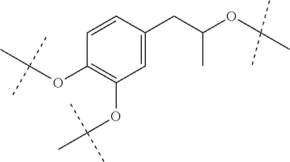 |
| L75 | 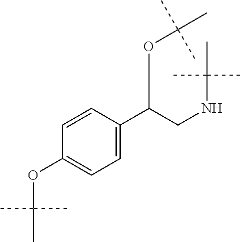 |
| L76 | 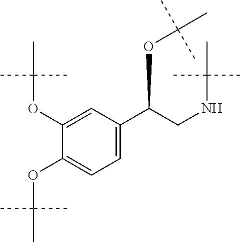 |
| L77 | 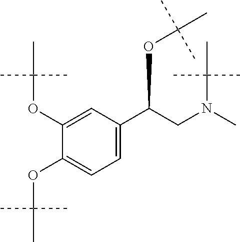 |
| L78 | 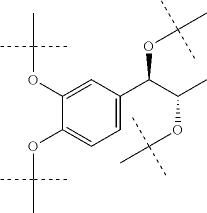 |
| L79 | 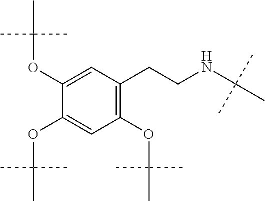 |
| L80 | 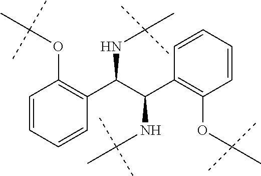 |
| L81 | 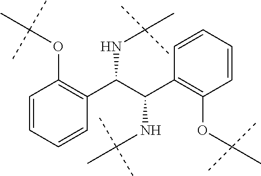 |
| L82 | 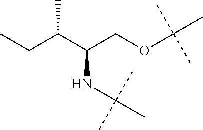 |
| L83 | 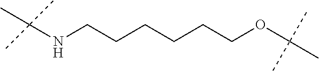 |
| L84 | 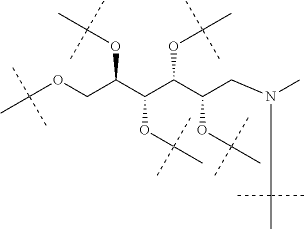 |
| L85 | 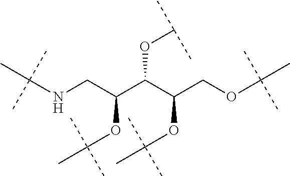 |
| L86 | 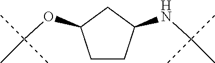 |
| L87 | 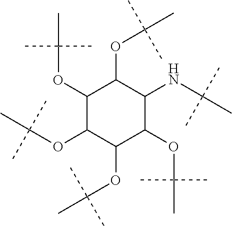 |
| L88 | 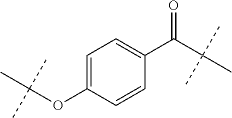 |
| L89 | 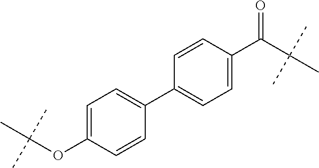 |
| L90 | 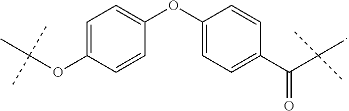 |

8. The senotherapeutic compound according to claim 1 for the treatment of one or more of the following disorders: Idiopathic pulmonary fibrosis (IPF), chronic obstructive pulmonary disease (COPD), asthma, cystic fibrosis, emphysema, bronchiectasis, and age-related loss of pulmonary function; Chronic kidney disease (CKD), interstitial nephritis, glomerulosclerosis/glomerulonephritis, acute kidney disease (AKD), kidney failure; Liver fibrosis, chronic hepatitis, non-alcoholic fatty liver disease (NAFLD); Pancreatic fibrosis, chronic pancreatitis; Myocardial fibrosis, infarction; Oral submucosa fibrosis; Neurodegenerative Diseases: Alzheimer's, Parkinson's, Multiple Sclerosis, mild cognitive impairment, motor neuron dysfunction, Huntington's disease, dementia; Neuropsychiatric disorders; Toxicity or inflammation, induced by chemotherapies, radiotherapies, HIV, stem cell transplant, other drugs; Osteoporosis, osteoarthritis, inflammatory bowel diseases (IBDs), inflammatory bowel syndrome (IBS), rheumatoid arthritis, oral mucositis, kyphosis, intervertebral disc degeneration, herniated intervertebral disc; Adipose atrophy; Sarcopenia, muscle/mobility loss due to aging, muscle fatigue; Atherosclerosis, angina, arrhythmia, cardiomyopathy, cardiomyocyte hypertrophy, congestive heart failure, coronary artery disease, carotid artery disease, endocarditis, coronary thrombosis, myocardial infarction, hypertension, aortic aneurysm, cardiac diastolic dysfunction, hypercholesterolemia, mitral valve prolapse, peripheral vascular disease, cardiac stress resistance, cardiac fibrosis, brain aneurysm, and stroke; Rare Diseases associated with aging and senescence, such as: aplastic anaemia, dyskeratosis congenita, Revetz syndrome, Hoyeraal-Hreidarsson syndrome, Lewy body dementia (LBD), amyloidosis, Paget's disease, diffuse idiopathic skeletal hyperostosis (DISH), multiple system atrophy (MSA); Diabetes (Type 2, Type 1), diabetic ulcer, obesity, metabolic syndrome; Wound healing; Frailty; Glaucoma, macular degeneration, cataracts, presbyopia, and vision loss; Hearing Loss; Immune function decline due to aging (Immunosenescence); Alopecia, Hair Loss; Melasma, discoloured skin, eczema, psoriasis, hyperpigmentation, nevi, rashes, atopic dermatitis, urticaria, diseases and disorders related to photosensitivity or photoaging, rhytides, pruritis, dysesthesia, eczematous eruptions, eosinophilic dermatosis, reactive neutrophilic dermatosis, pemphigoidus dermatosis, fibrohistocytic proliferations of skin, cutaneous lymphomas, and cutaneous lupus; Transplant of kidney, liver, lung, heart, pancreas or other organ.

9. A senolytic compound, comprising a senotherapeutic compound according to claim 1.

10. The senotherapeutic compound according to claim 1 in combination with other known drugs of the type selected from; senolytics, senomorphic, senostatic, senotherapeutic, cellular senescence promoters, and compounds that preserve tissue integrity for the production of a senotherapeutic drug for the treatment of one or more of the following disorders; Idiopathic pulmonary fibrosis (IPF), chronic obstructive pulmonary disease (COPD), asthma, cystic fibrosis, emphysema, bronchiectasis, and age-related loss of pulmonary function; Chronic kidney disease (CKD), interstitial nephritis, glomerulosclerosis/glomerulonephritis, acute kidney disease (AKD), kidney failure; Liver fibrosis, chronic hepatitis, non-alcoholic fatty liver disease (NAFLD); Pancreatic fibrosis, chronic pancreatitis; Myocardial fibrosis, infarction; Oral submucosa fibrosis; Neurodegenerative Diseases; Alzheimer's, Parkinson's, Multiple Sclerosis, mild cognitive impairment, motor neuron dysfunction, Huntington's disease, dementia; Neuropsychiatric disorders; Toxicity or inflammation, induced by chemotherapies, radiotherapies, HIV, stem cell transplant, other drugs; Osteoporosis, osteoarthritis, inflammatory bowel diseases (IBDs), inflammatory bowel syndrome (IBS), rheumatoid arthritis, oral mucositis, kyphosis, intervertebral disc degeneration, herniated intervertebral disc; Adipose atrophy; Sarcopenia, muscle/mobility loss due to aging, muscle fatigue; Atherosclerosis, angina, arrhythmia, cardiomyopathy, cardiomyocyte hypertrophy, congestive heart failure, coronary artery disease, carotid artery disease, endocarditis, coronary thrombosis, myocardial infarction, hypertension, aortic aneurysm, cardiac diastolic dysfunction, hypercholesterolemia, mitral valve prolapse, peripheral vascular disease, cardiac stress resistance, cardiac fibrosis, brain aneurysm, and stroke; Rare Diseases associated with aging and senescence, such as; aplastic anaemia, dyskeratosis congenita, Revetz syndrome, Hoyeraal-Hreidarsson syndrome, Lewy body dementia (LBD), amyloidosis, Paget's disease, diffuse idiopathic skeletal hyperostosis (DISH), multiple system atrophy (MSA); Diabetes (Type 2, Type 1), diabetic ulcer, obesity, metabolic syndrome; Wound healing; Frailty; Glaucoma, macular degeneration, cataracts, presbyopia, and vision loss; Hearing Loss; Immune function decline due to aging (Immunosenescence), Alopecia, Hair Loss; Melasma, discoloured skin, eczema, psoriasis, hyperpigmentation, nevi, rashes, atopic dermatitis, urticaria, diseases and disorders related to photosensitivity or photoaging, rhytides, pruritis, dysesthesia, eczematous eruptions, eosinophilic dermatosis, reactive neutrophilic dermatosis, pemphigoidus dermatosis, fibrohistocytic proliferations of skin, cutaneous lymphomas, and cutaneous lupus; Transplant of kidney, liver, lung, heart, pancreas or other organ.

11. The senotherapeutic compound according to claim 6, wherein R′₁ is a Linker L and said linker is selected from the following compounds:

| LINKER L | |
|---|---|
| L1 | 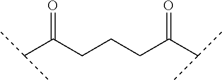 |
| L2 | 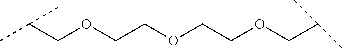 |
| L3 | 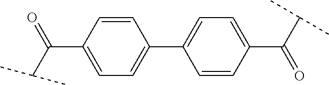 |
| L4 | 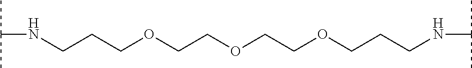 |
| L5 | 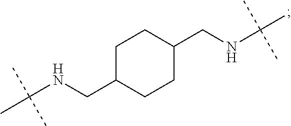 |
| | 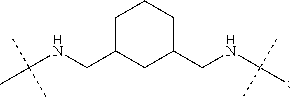 |
| | 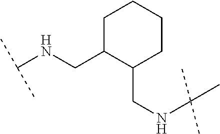 |
| L6 | 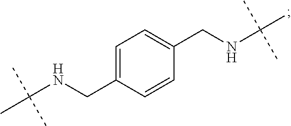 |
| | 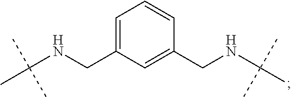 |
| | 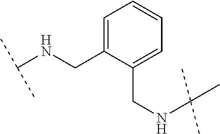 |
| L7 | 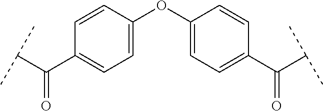 |
| L8 | 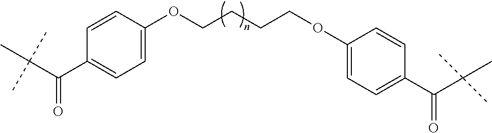 |
| | n = 0-8 |
| L9 | 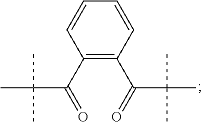 |
| | 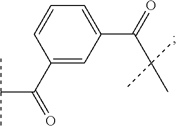 |
| | 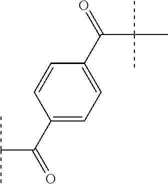 |
| L11 | 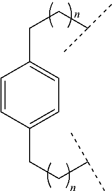 |
| | n = 1-6, each independently |
| L12 | 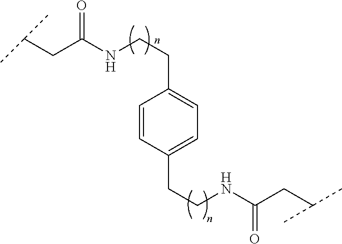 |
| | n = 1-6 |
| L13 | 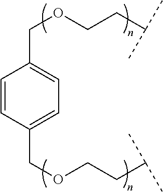 |
| | n = 1-3, each independently |
| L14 | 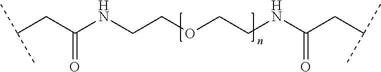 |
| | n = 1-4 |
| L15 | 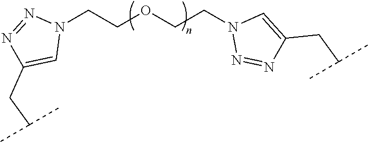 |
| | n = 1-4, each independently |
| L16 | 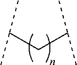 |
| | n = 2-12 |
| L17 | 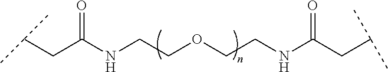 |
| | n = 1-5 |
| L18 | 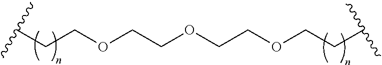 |
| | n = 1-3 |
| L19 | 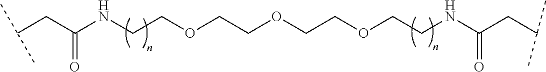 |
| | n = 1-3 |
| L20 | 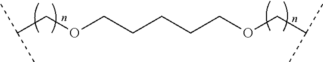 |
| | n = 1-3 |
| L21 | 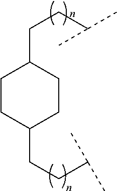 |
| | n = 1-6 |
| L22 | 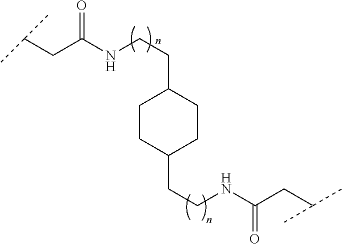 |
| | n = 1-6 |
| L23 | 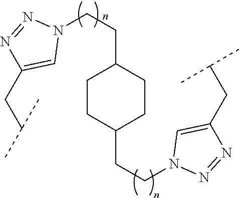 |
| | n = 1-6, each independently |
| L24 | 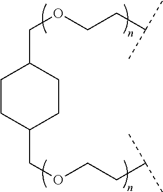 |
| | n = 1-3, each independently |
| L25 | 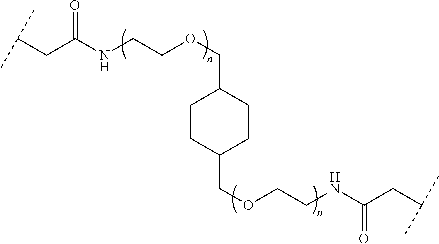 |
| | n = 1-3, each independently |
| L26 | 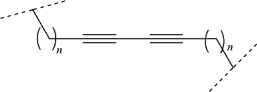 |
| | n = 1-6, each independently |
| L27 | 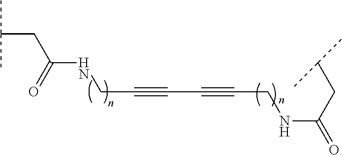 |
| | n = 1-6, each independently |
| L28 | 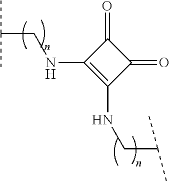 |
| | n = 1-6, each independently |
| L29 | 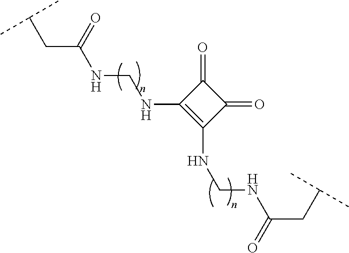 |
| | n = 1-6, each independently |
| L30 | 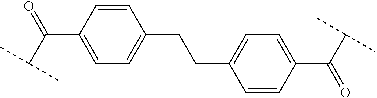 |

12. The senotherapeutic compound according to claim 6, wherein R′₁ is a Linker L and said linker is selected from the following compounds:

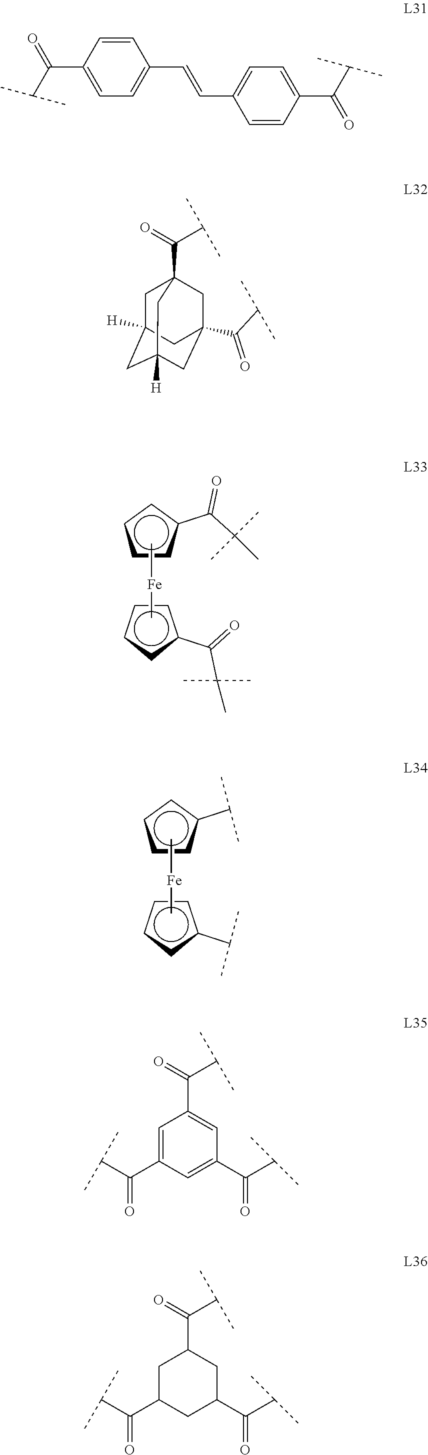

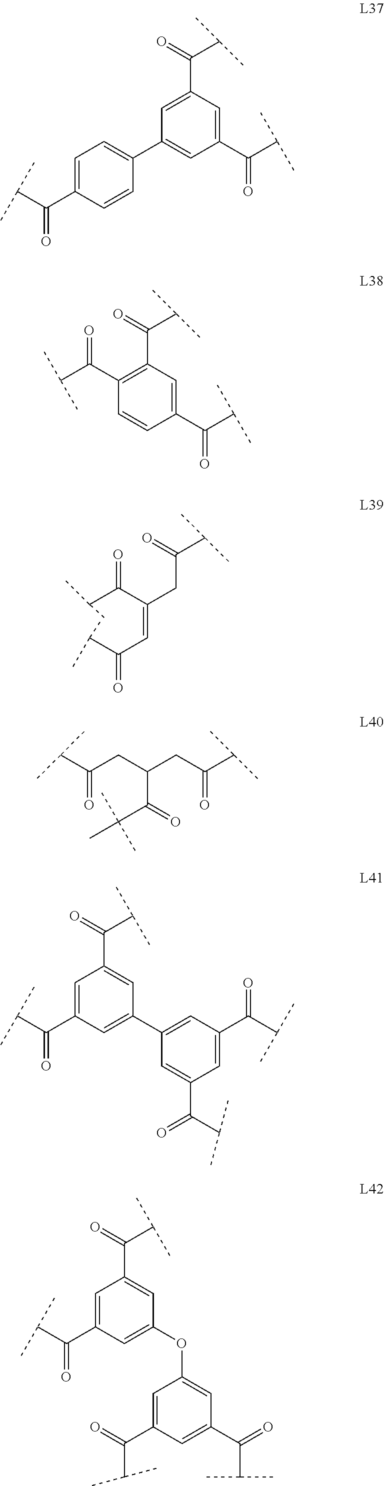

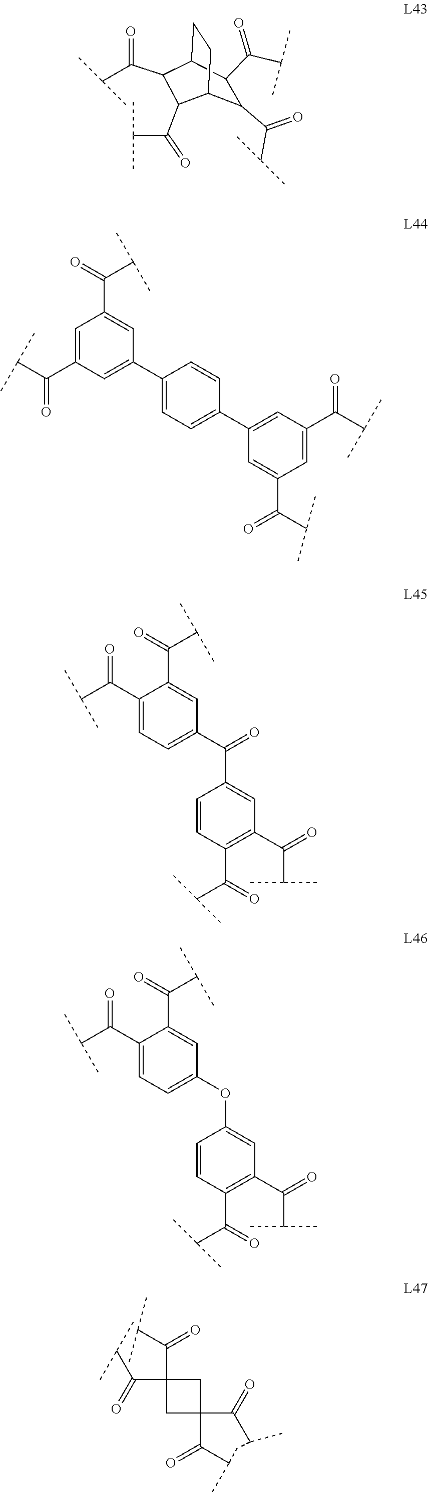

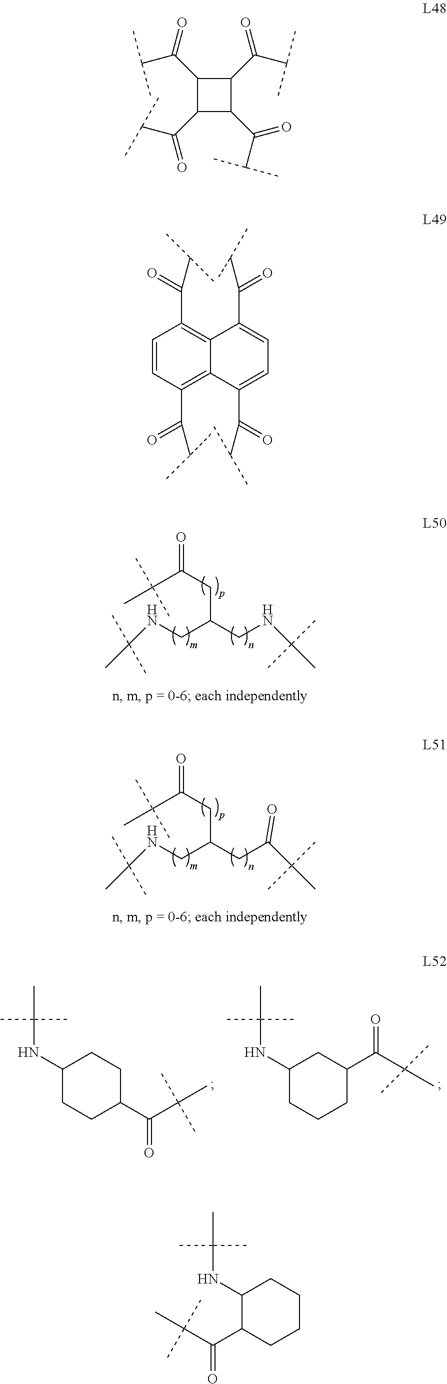

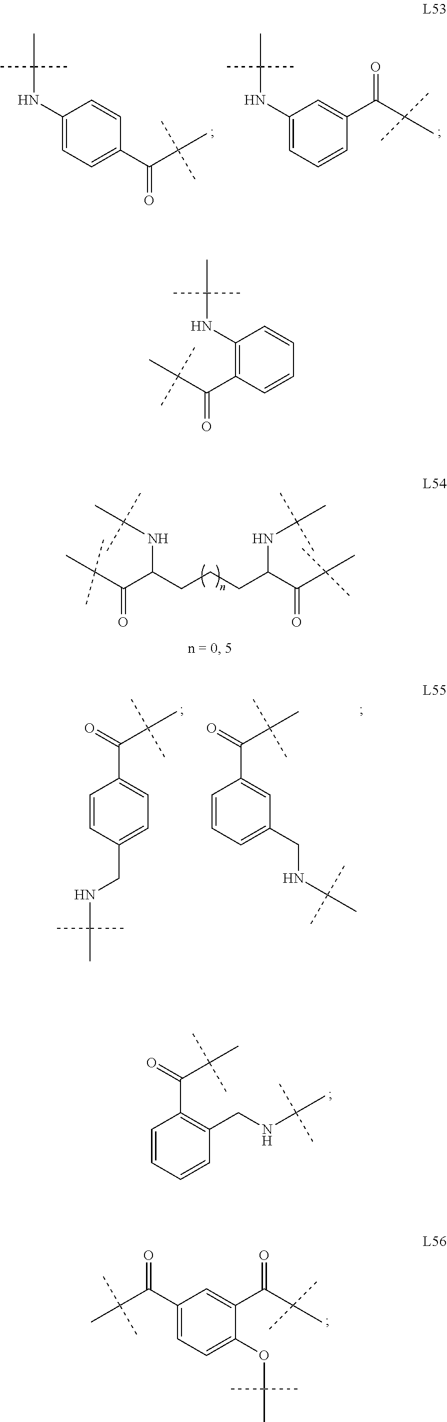

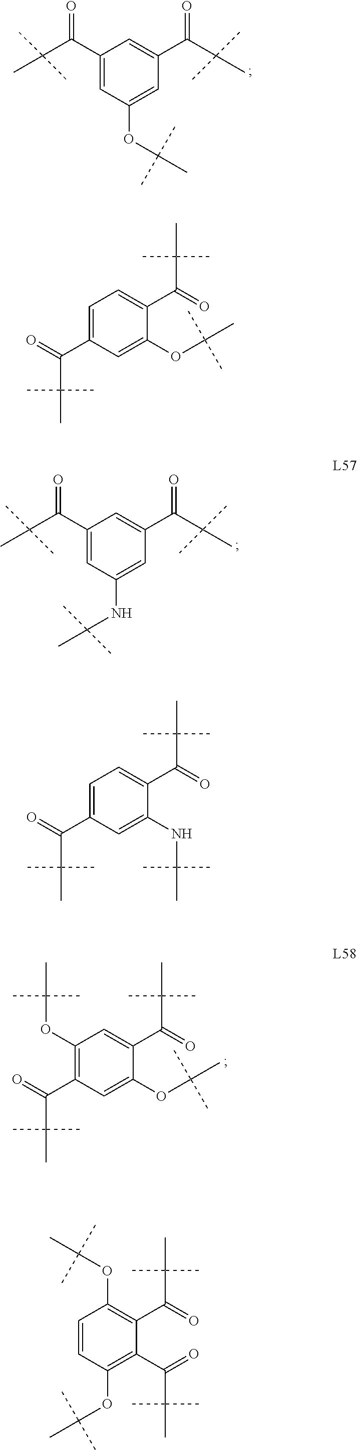

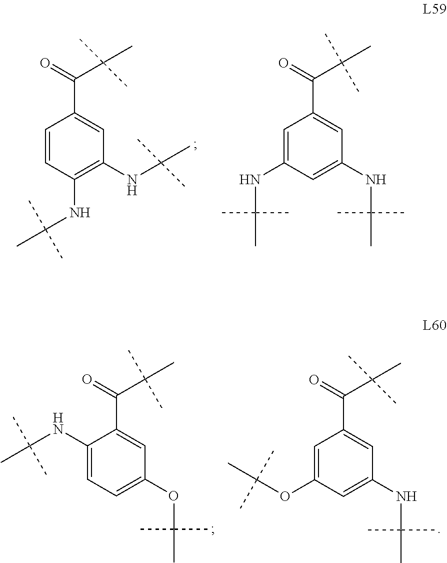

13. The senotherapeutic compound according to claim 6, wherein R′₁ is a Linker L and said linker is selected from the following compounds:

| LINKER L | |
|---|---|
| L61 | 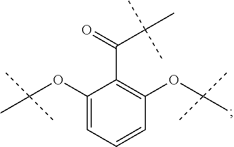 |
| | 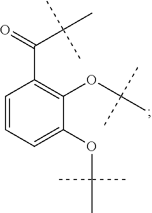 |
| | 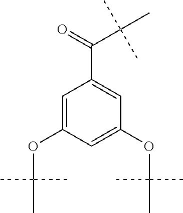 |
| L62 | 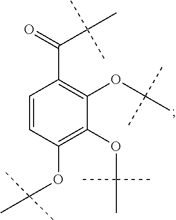 |
| | 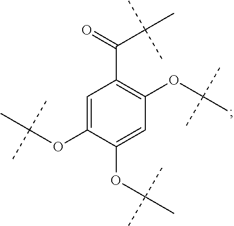 |
| L63 | 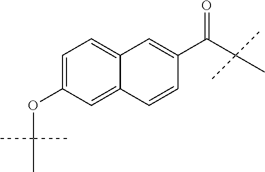 |
| L64 | 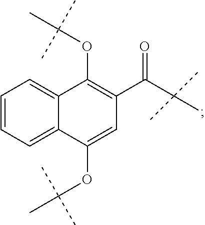 |
| | 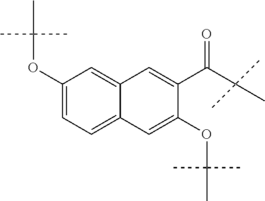 |
| L65 | 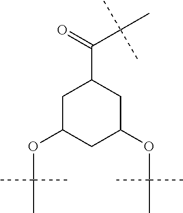 |
| L66 | 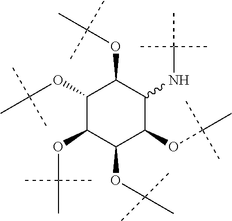 |
| L67 | 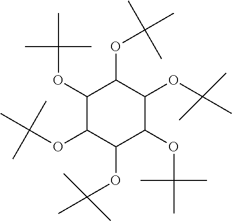 [SMILES: CC(C)(C)OC1C(OC(C)(C)C)C(OC(C)(C)C)C(OC(C)(C)C)C(OC(C)(C)C)C1OC(C)(C)C] |
| L68 | 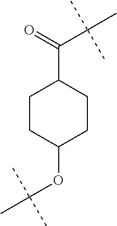 |
| L69 | 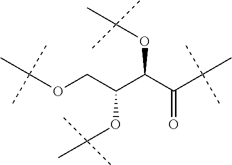 |
| L70 | 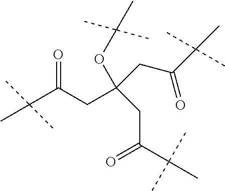 |
| L71 | 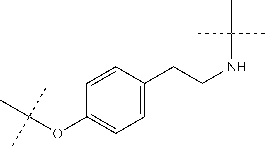 |
| L72 | 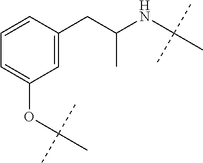 |
| L73 | 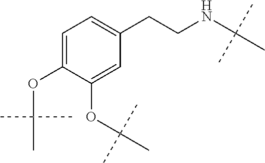 |
| L74 | 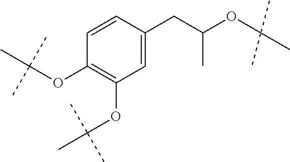 |
| L75 | 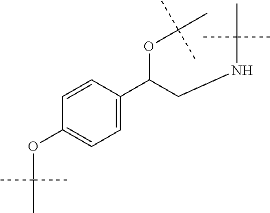 |
| L76 | 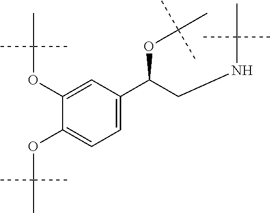 |
| L77 | 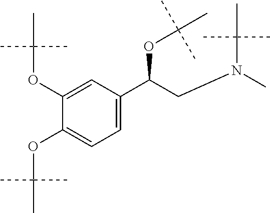 |
| L78 | 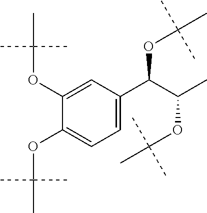 |
| L79 | 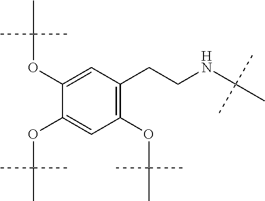 |
| L80 | 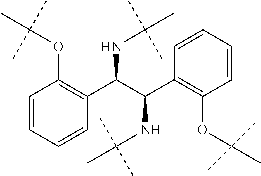 |
| L81 | 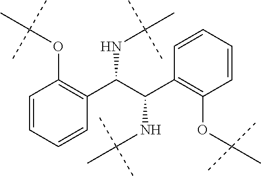 |
| L82 | 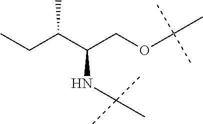 |
| L83 | 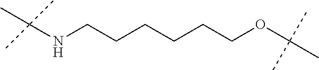 |
| L84 | 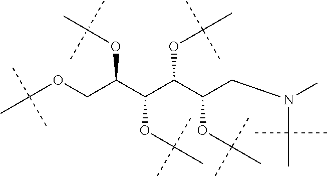 |
| L85 | 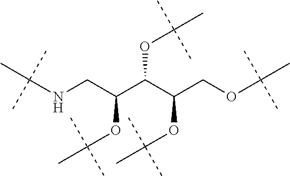 |
| L86 | 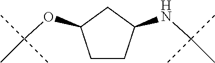 |
| L87 | 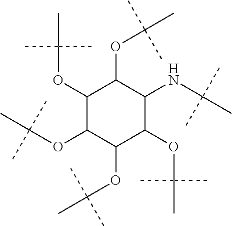 |
| L88 | 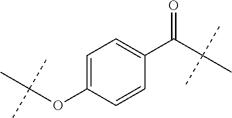 |
| L89 | 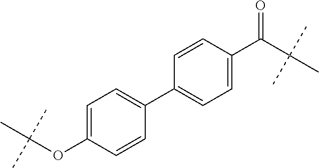 |
| L90 | 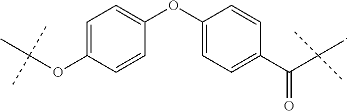 |

14. The senotherapeutic compound according to claim 1 for the treatment of one or more of the following disorders: Idiopathic pulmonary fibrosis (IPF), chronic obstructive pulmonary disease (COPD), asthma, cystic fibrosis, emphysema, bronchiectasis, and age-related loss of pulmonary function; Chronic kidney disease (CKD), interstitial nephritis, glomerulosclerosis/glomerulonephritis, acute kidney disease (AKD), kidney failure; Liver fibrosis, chronic hepatitis, non-alcoholic fatty liver disease (NAFLD); Pancreatic fibrosis, chronic pancreatitis; Myocardial fibrosis, infarction; Oral submucosa fibrosis; Neurodegenerative Diseases: Alzheimer's, Parkinson's, Multiple Sclerosis, mild cognitive impairment, motor neuron dysfunction, Huntington's disease, dementia; Neuropsychiatric disorders; Toxicity or inflammation, induced by chemotherapies, radiotherapies, HIV, stem cell transplant, other drugs.

15. The senotherapeutic compound according to claim 1 for the treatment of one or more of the following disorders: Osteoporosis, osteoarthritis, inflammatory bowel diseases (IBDs), inflammatory bowel syndrome (IBS), rheumatoid arthritis, oral mucositis, kyphosis, intervertebral disc degeneration, herniated intervertebral disc; Adipose atrophy; Sarcopenia, muscle/mobility loss due to aging, muscle fatigue; Atherosclerosis, angina, arrhythmia, cardiomyopathy, cardiomyocyte hypertrophy, congestive heart failure, coronary artery disease, carotid artery disease, endocarditis, coronary thrombosis, myocardial infarction, hypertension, aortic aneurysm, cardiac diastolic dysfunction, hypercholesterolemia, mitral valve prolapse, peripheral vascular disease, cardiac stress resistance, cardiac fibrosis, brain aneurysm, and stroke; Rare Diseases associated with aging and senescence, such as: aplastic anaemia, dyskeratosis congenita, Revetz syndrome, Hoyeraal-Hreidarsson syndrome, Lewy body dementia (LBD), amyloidosis, Paget's disease, diffuse idiopathic skeletal hyperostosis (DISH), multiple system atrophy (MSA; Diabetes (Type 2, Type 1), diabetic ulcer, obesity, metabolic syndrome;

16. The senotherapeutic compound according to claim 1 for the treatment of one or more of the following disorders: multiple system atrophy (MSA; Diabetes (Type 2, Type 1), diabetic ulcer, obesity, metabolic syndrome, Wound healing; Frailty; Glaucoma, macular degeneration, cataracts, presbyopia, and vision loss; Hearing Loss; Immune function decline due to aging (Immunosenescence); Alopecia, Hair Loss; Melasma, discoloured skin, eczema, psoriasis, hyperpigmentation, nevi, rashes, atopic dermatitis, urticaria, diseases and disorders related to photosensitivity or photoaging, rhytides, pruritis, dysesthesia, eczematous eruptions, eosinophilic dermatosis, reactive neutrophilic dermatosis, pemphigoidus dermatosis, fibrohistocytic proliferations of skin, cutaneous lymphomas, and cutaneous lupus; Transplant of kidney, liver, lung, heart, pancreas or other organ.

17. A method of use of the senotherapeutic compound according to claim 1, comprising administering the senotherapeutic compound to a person for eliminating senescent cells, or suppressing the phenotype of senescent cells, for the treatment of one or more of the following disorders: Idiopathic pulmonary fibrosis (IPF), chronic obstructive pulmonary disease (COPD), asthma, cystic fibrosis, emphysema, bronchiectasis, and age-related loss of pulmonary function; Chronic kidney disease (CKD), interstitial nephritis, glomerulosclerosis/glomerulonephritis, acute kidney disease (AKD), kidney failure; Liver fibrosis, chronic hepatitis, non-alcoholic fatty liver disease (NAFLD); Pancreatic fibrosis, chronic pancreatitis; Myocardial fibrosis, infarction; Oral submucosa fibrosis; Neurodegenerative Diseases: Alzheimer's, Parkinson's, Multiple Sclerosis, mild cognitive impairment, motor neuron dysfunction, Huntington's disease, dementia; Neuropsychiatric disorders; Toxicity or inflammation, induced by chemotherapies, radiotherapies, HIV, stem cell transplant, other drugs-etc-; Osteoporosis, osteoarthritis, inflammatory bowel diseases (IBDs), inflammatory bowel syndrome (IBS), rheumatoid arthritis, oral mucositis, kyphosis, intervertebral disc degeneration, herniated intervertebral disc; Adipose atrophy; Sarcopenia, muscle/mobility loss due to aging, muscle fatigue; Atherosclerosis, angina, arrhythmia, cardiomyopathy, cardiomyocyte hypertrophy, congestive heart failure, coronary artery disease, carotid artery disease, endocarditis, coronary thrombosis, myocardial infarction, hypertension, aortic aneurysm, cardiac diastolic dysfunction, hypercholesterolemia, mitral valve prolapse, peripheral vascular disease, cardiac stress resistance, cardiac fibrosis, brain aneurysm, and stroke; Rare Diseases associated with aging and senescence, such as: aplastic anaemia, dyskeratosis congenita, Revetz syndrome, Hoyeraal-Hreidarsson syndrome, Lewy body dementia (LBD), amyloidosis, Paget's disease, diffuse idiopathic skeletal hyperostosis (DISH), multiple system atrophy (MSA); Diabetes (Type 2, Type 1), diabetic ulcer, obesity, metabolic syndrome; Wound healing; Frailty; Glaucoma, macular degeneration, cataracts, presbyopia, and vision loss; Hearing Loss; Immune function decline due to aging (Immunosenescence); Alopecia, Hair Loss; Melasma, discoloured skin, eczema, psoriasis, hyperpigmentation, nevi, rashes, atopic dermatitis, urticaria, diseases and disorders related to photosensitivity or photoaging, rhytides, pruritis, dysesthesia, eczematous eruptions, eosinophilic dermatosis, reactive neutrophilic dermatosis, pemphigoidus dermatosis, fibrohistocytic proliferations of skin, cutaneous lymphomas, and cutaneous lupus; Transplant of kidney, liver, lung, heart, pancreas or other organ.

18. A method of use of the senotherapeutic compound according to claim 1, comprising administering the senotherapeutic compound in combination with other known drugs of the type selected from: senolytics, senomorphic, senostatic, senotherapeutic, cellular senescence promoters, and compounds that preserve tissue integrity for the production of a senotherapeutic drug, to a person for eliminating senescent cells, or suppressing the phenotype of senescent cells, for treatment of one or more of the following disorders: Idiopathic pulmonary fibrosis (IPF), chronic obstructive pulmonary disease (COPD), asthma, cystic fibrosis, emphysema, bronchiectasis, and age-related loss of pulmonary function; Chronic kidney disease (CKD), interstitial nephritis, glomerulosclerosis/glomerulonephritis, acute kidney disease (AKD), kidney failure; Liver fibrosis, chronic hepatitis, non-alcoholic fatty liver disease (NAFLD); Pancreatic fibrosis, chronic pancreatitis; Myocardial fibrosis, infarction; Oral submucosa fibrosis; Neurodegenerative Diseases: Alzheimer's, Parkinson's, Multiple Sclerosis, mild cognitive impairment, motor neuron dysfunction, Huntington's disease, dementia; Neuropsychiatric disorders; Toxicity or inflammation, induced by chemotherapies, radiotherapies, HIV, stem cell transplant, other drugs; Osteoporosis, osteoarthritis, inflammatory bowel diseases (IBDs), inflammatory bowel syndrome (IBS), rheumatoid arthritis, oral mucositis, kyphosis, intervertebral disc degeneration, herniated intervertebral disc; Adipose atrophy; Sarcopenia, muscle/mobility loss due to aging, muscle fatigue; Atherosclerosis, angina, arrhythmia, cardiomyopathy, cardiomyocyte hypertrophy, congestive heart failure, coronary artery disease, carotid artery disease, endocarditis, coronary thrombosis, myocardial infarction, hypertension, aortic aneurysm, cardiac diastolic dysfunction, hypercholesterolemia, mitral valve prolapse, peripheral vascular disease, cardiac stress resistance, cardiac fibrosis, brain aneurysm, and stroke; Rare Diseases associated with aging and senescence, such as: aplastic anaemia, dyskeratosis congenita, Revetz syndrome, Hoyeraal-Hreidarsson syndrome, Lewy body dementia (LBD), amyloidosis, Paget's disease, diffuse idiopathic skeletal hyperostosis (DISH), multiple system atrophy (MSA); Diabetes (Type 2, Type 1), diabetic ulcer, obesity, metabolic syndrome; Wound healing; Frailty; Glaucoma, macular degeneration, cataracts, presbyopia, and vision loss; Hearing Loss; Immune function decline due to aging (Immunosenescence); Alopecia, Hair Loss; Melasma, discoloured skin, eczema, psoriasis, hyperpigmentation, nevi, rashes, atopic dermatitis, urticaria, diseases and disorders related to photosensitivity or photoaging, rhytides, pruritis, dysesthesia, eczematous eruptions, eosinophilic dermatosis, reactive neutrophilic dermatosis, pemphigoidus dermatosis, fibrohistocytic proliferations of skin, cutaneous lymphomas, and cutaneous lupus; Transplant of kidney, liver, lung, heart, pancreas or other organ.

19. A composition comprising the senotherapeutic compound according to claim 1, for eliminating senescent cells, or suppressing the phenotype of senescent cells, for treatment of one or more of the following disorders: Idiopathic pulmonary fibrosis (IPF), chronic obstructive pulmonary disease (COPD), asthma, cystic fibrosis, emphysema, bronchiectasis, and age-related loss of pulmonary function; Chronic kidney disease (CKD), interstitial nephritis, glomerulosclerosis/glomerulonephritis, acute kidney disease (AKD), kidney failure; Liver fibrosis, chronic hepatitis, non-alcoholic fatty liver disease (NAFLD); Pancreatic fibrosis, chronic pancreatitis; Myocardial fibrosis, infarction; Oral submucosa fibrosis; Neurodegenerative Diseases: Alzheimer's, Parkinson's, Multiple Sclerosis, mild cognitive impairment, motor neuron dysfunction, Huntington's disease, dementia; Neuropsychiatric disorders; Toxicity or inflammation, induced by chemotherapies, radiotherapies, HIV, stem cell transplant, other drugs; Osteoporosis, osteoarthritis, inflammatory bowel diseases (IBDs), inflammatory bowel syndrome (IBS), rheumatoid arthritis, oral mucositis, kyphosis, intervertebral disc degeneration, herniated intervertebral disc; Adipose atrophy; Sarcopenia, muscle/mobility loss due to aging, muscle fatigue; Atherosclerosis, angina, arrhythmia, cardiomyopathy, cardiomyocyte hypertrophy, congestive heart failure, coronary artery disease, carotid artery disease, endocarditis, coronary thrombosis, myocardial infarction, hypertension, aortic aneurysm, cardiac diastolic dysfunction, hypercholesterolemia, mitral valve prolapse, peripheral vascular disease, cardiac stress resistance, cardiac fibrosis, brain aneurysm, and stroke; Rare Diseases associated with aging and senescence, such as: aplastic anaemia, dyskeratosis congenita, Revetz syndrome, Hoyeraal-Hreidarsson syndrome, Lewy body dementia (LBD), amyloidosis, Paget's disease, diffuse idiopathic skeletal hyperostosis (DISH), multiple system atrophy (MSA); Diabetes (Type 2, Type 1), diabetic ulcer, obesity, metabolic syndrome; Wound healing; Frailty; Glaucoma, macular degeneration, cataracts, presbyopia, and vision loss; Hearing Loss; Immune function decline due to aging (Immunosenescence); Alopecia, Hair Loss; Melasma, discoloured skin, eczema, psoriasis, hyperpigmentation, nevi, rashes, atopic dermatitis, urticaria, diseases and disorders related to photosensitivity or photoaging, rhytides, pruritis, dysesthesia, eczematous eruptions, eosinophilic dermatosis, reactive neutrophilic dermatosis, pemphigoidus dermatosis, fibrohistocytic proliferations of skin, cutaneous lymphomas, and cutaneous lupus; Transplant of kidney, liver, lung, heart, pancreas or other organ

20. A composition comprising the senotherapeutic compound according to claim 1 in combination with other known drugs of the type selected from: senolytics, senomorphic, senostatic, senotherapeutic, cellular senescence promoters, and compounds that preserve tissue integrity, for eliminating senescent cells, or suppressing the phenotype of senescent cells, for treatment of one or more of the following disorders: Idiopathic pulmonary fibrosis (IPF), chronic obstructive pulmonary disease (COPD), asthma, cystic fibrosis, emphysema, bronchiectasis, and age-related loss of pulmonary function; Chronic kidney disease (CKD), interstitial nephritis, glomerulosclerosis/glomerulonephritis, acute kidney disease (AKD), kidney failure; Liver fibrosis, chronic hepatitis, non-alcoholic fatty liver disease (NAFLD); Pancreatic fibrosis, chronic pancreatitis; Myocardial fibrosis, infarction; Oral submucosa fibrosis; Neurodegenerative Diseases: Alzheimer's, Parkinson's, Multiple Sclerosis, mild cognitive impairment, motor neuron dysfunction, Huntington's disease, dementia; Neuropsychiatric disorders; Toxicity or inflammation, induced by chemotherapies, radiotherapies, HIV, stem cell transplant, other drugs; Osteoporosis, osteoarthritis, inflammatory bowel diseases (IBDs), inflammatory bowel syndrome (IBS), rheumatoid arthritis, oral mucositis, kyphosis, intervertebral disc degeneration, herniated intervertebral disc; Adipose atrophy; Sarcopenia, muscle/mobility loss due to aging, muscle fatigue; Atherosclerosis, angina, arrhythmia, cardiomyopathy, cardiomyocyte hypertrophy, congestive heart failure, coronary artery disease, carotid artery disease, endocarditis, coronary thrombosis, myocardial infarction, hypertension, aortic aneurysm, cardiac diastolic dysfunction, hypercholesterolemia, mitral valve prolapse, peripheral vascular disease, cardiac stress resistance, cardiac fibrosis, brain aneurysm, and stroke; Rare Diseases associated with aging and senescence, such as: aplastic anaemia, dyskeratosis congenita, Revetz syndrome, Hoyeraal-Hreidarsson syndrome, Lewy body dementia (LBD), amyloidosis, Paget's disease, diffuse idiopathic skeletal hyperostosis (DISH), multiple system atrophy (MSA); Diabetes (Type 2, Type 1), diabetic ulcer, obesity, metabolic syndrome; Wound healing; Frailty; Glaucoma, macular degeneration, cataracts, presbyopia, and vision loss; Hearing Loss; Immune function decline due to aging (Immunosenescence); Alopecia, Hair Loss; Melasma, discoloured skin, eczema, psoriasis, hyperpigmentation, nevi, rashes, atopic dermatitis, urticaria, diseases and disorders related to photosensitivity or photoaging, rhytides, pruritis, dysesthesia, eczematous eruptions, eosinophilic dermatosis, reactive neutrophilic dermatosis, pemphigoidus dermatosis, fibrohistocytic proliferations of skin, cutaneous lymphomas, and cutaneous lupus; Transplant of kidney, liver, lung, heart, pancreas or other organ
